SEQUENCE LISTING (77 sequences)

SEQ ID NO: 1 - protein, 469 aa, Artificial Sequence
  DiaL19
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 2 - protein, 477 aa, Artificial Sequence
  DiaF8
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 3 - protein, 475 aa, Artificial Sequence
  DiaNHS76
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 4 - protein, 476 aa, Artificial Sequence
  DiaNHS76F8
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 5 - protein, 472 aa, Artificial Sequence
  DiaNHS76L19
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 6 - protein, 476 aa, Artificial Sequence
  DiaF8NHS76
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 7 - protein, 473 aa, Artificial Sequence
  DiaF8L19
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 8 - protein, 472 aa, Artificial Sequence
  DiaL19NHS76
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 9 - protein, 473 aa, Artificial Sequence
  DiaL19F8
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 10 - protein, 108 aa, Artificial Sequence [sequence omitted]

SEQ ID NO: 11 - protein, 114 aa, Artificial Sequence
  L19VH
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTV

SEQ ID NO: 12 - protein, 108 aa, Artificial Sequence
  F8VL
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 13 - protein, 118 aa, Artificial Sequence
  F8VH
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 14 - protein, 108 aa, Artificial Sequence
  NHS76VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 15 - protein, 117 aa, Artificial Sequence
  NHS76VH
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSS

SEQ ID NO: 16 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 17 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 18 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 19 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 20 - protein, 124 aa, Rattus norvegicus
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 21 - protein, 127 aa, Homo sapiens [sequence omitted]

SEQ ID NO: 22 - protein, 15 aa, Artificial Sequence
  Linker 1
GGGGSGGGGSGGGGS

SEQ ID NO: 23 - protein, 16 aa, Artificial Sequence
  Linker 2
GGGGSLVPRGSGGGGS

SEQ ID NO: 24 - protein, 15 aa, Artificial Sequence
  Linker 3
SSSSGSSSSGSSSSG

SEQ ID NO: 25 - protein, 15 aa, Artificial Sequence
  Linker 4
SSSSGLVPRGSSSSG

SEQ ID NO: 26 - protein, 6 aa, Artificial Sequence
  Linker 5
GSADGG

SEQ ID NO: 27 - protein, 824 aa, Artificial Sequence
  mIL12bIL12aIL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 28 - protein, 824 aa, Artificial Sequence
  mIL12bIL12aGMCSFIL2
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 29 - protein, 808 aa, Artificial Sequence
  hIL12bIL12aIL2GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 30 - protein, 1308 aa, Artificial Sequence
  mIL12bIL12aDiaL19IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 31 - protein, 1316 aa, Artificial Sequence
  mIL12bIL12aDiaF8IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 32 - protein, 1314 aa, Artificial Sequence
  mIL12bIL12aDiaNHS76IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 33 - protein, 1315 aa, Artificial Sequence
  mIL12bIL12aDiaNHS76F8IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 34 - protein, 1311 aa, Artificial Sequence
  mIL12bIL12aDiaNHS76L19IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 35 - protein, 1315 aa, Artificial Sequence
  mIL12bIL12aDiaF8NHS76IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 36 - protein, 1312 aa, Artificial Sequence
  mIL12bIL12aDiaF8L19IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 37 - protein, 1311 aa, Artificial Sequence
  mIL12bIL12aDiaL19NHS76IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 38 - protein, 1312 aa, Artificial Sequence
  mIL12bIL12aDiaL19F8IL2GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 39 - protein, 1315 aa, Artificial Sequence
  mIL12bIL12aIL2DiaNHS76F8GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 40 - protein, 1316 aa, Artificial Sequence
  mIL12bIL12aIL2DiaF8GMCSF
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 41 - protein, 1315 aa, Artificial Sequence
  mIL12bIL12aIL2GMCSFDiaNHS76F8
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 42 - protein, 1316 aa, Artificial Sequence
  mIL12bIL12aIL2GMCSFDiaF8
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 43 - protein, 1292 aa, Artificial Sequence
  hIL12bIL12aDiaL19IL2GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSSSSGSSSSGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 44 - protein, 1298 aa, Artificial Sequence
  hIL12bIL12aDiaNHS76IL2GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSSSSGSSSSGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 45 - protein, 1299 aa, Artificial Sequence
  hIL12bIL12aDiaNHS76F8IL2GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSSSSGSSSSGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 46 - protein, 1299 aa, Artificial Sequence
  hIL12bIL12aIL2DiaNHS76F8GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 47 - protein, 1300 aa, Artificial Sequence
  hIL12bIL12aIL2DiaF8GMCSF
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 48 - protein, 1292 aa, Artificial Sequence
  hIL12bIL12aDiaL19GMCSFIL2
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSSSSGSSSSGSSSSGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVGGSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQESSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 49 - protein, 1298 aa, Artificial Sequence
  hIL12bIL12aDiaNHS76GMCSFIL2
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGSSSSGSSSSGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQESSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 50 - protein, 1299 aa, Artificial Sequence
  hIL12bIL12aDiaNHS76F8GMCSFIL2
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQESSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 51 - protein, 1316 aa, Artificial Sequence
  mIL12bIL12aIL2DiaNHS76F8GMCSF-Thr
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSLVPRGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGLVPRGSSSSGAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 52 - protein, 1300 aa, Artificial Sequence
  hIL12bIL12aIL2DiaNHS76F8GMCSF-Thr
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSLVPRGSGGGGSQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSGGSGGEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKSSSSGSSSSGSSSSGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSGGSGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLSSSSGLVPRGSSSSGAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 53 - protein, 357 aa, Artificial Sequence
  First polypeptide chain (i.e. mouse mil12ail2 chain)
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 54 - protein, 357 aa, Artificial Sequence
  First polypeptide chain (i.e. mouse mil2il12a chain)
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 55 - protein, 332 aa, Artificial Sequence
  First polypeptide chain (i.e. mouse mil12agmcsf chain)
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 56 - protein, 345 aa, Artificial Sequence
  First polypeptide chain (i.e. human hil12ail2 chain)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 57 - protein, 452 aa, Artificial Sequence
  Second polypeptide chain (i.e. mouse mil12bgmcsf chain)
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 58 - protein, 452 aa, Artificial Sequence
  Second polypeptide chain (i.e. mouse mgmcsfil12b chain)
APTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQKGGGGSGGGGSGGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 59 - protein, 477 aa, Artificial Sequence
  Second polypeptide chain (i.e. mouse mil12bil2 chain)
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 60 - protein, 448 aa, Artificial Sequence
  Second polypeptide chain (i.e. human hil12bgmcsf chain)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 61 - protein, 555 aa, Artificial Sequence
  Second polypeptide chain (i.e. mouse mil12bscl19 chain)
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSADGGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 62 - protein, 559 aa, Artificial Sequence
  Second polypeptide chain (i.e. mouse mil12bscf8 chain)
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSADGGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSSSGDGSSGGSGGASEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 63 - protein, 558 aa, Artificial Sequence
  Second polypeptide chain (i.e. mouse mil12bscnhs76 chain)
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGSADGGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSSSGDGSSGGSGGASSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 64 - protein, 552 aa, Artificial Sequence
  Second polypeptide chain (i.e. human hil12bscf8 chain)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSADGGEVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSSSGDGSSGGSGGASEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 65 - protein, 551 aa, Artificial Sequence
  Second polypeptide chain (i.e. human hil12bscnhs76 chain)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGSADGGQVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSSSGDGSSGGSGGASSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 66 - protein, 496 aa, Artificial Sequence
  The first polypeptide chain (i.e. mouse mil2il12agmcsf chain)
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 67 - protein, 738 aa, Artificial Sequence
  First polypeptide chain (i.e. mouse mscl19il12ail2gmcsf chain)
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGSADGGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 68 - protein, 742 aa, Artificial Sequence
  First polypeptide chain (i.e. mouse mscf8il12ail2gmcsf chain)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSSSGDGSSGGSGGASEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGSADGGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 69 - protein, 741 aa, Artificial Sequence
  First polypeptide chain (i.e. mouse mscnhs76il12ail2gmcsf chain)
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSSSGDGSSGGSGGASSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGSADGGRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAGGGGSGGGGSGGGGSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 70 - protein, 487 aa, Artificial Sequence
  The first polypeptide chain (i.e. human hil2il12agmcsf chain)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 71 - protein, 733 aa, Artificial Sequence
  First polypeptide chain (i.e. human hscf8il12ail2gmcsf chain)
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSSSSGDGSSGGSGGASEIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIKGSADGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 72 - protein, 732 aa, Artificial Sequence
  First polypeptide chain (i.e. human hscnhs76il12ail2gmcsf chain)
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCARGKWSKFDYWGQGTLVTVSSSSGDGSSGGSGGASSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGSADGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 73 - DNA, 2613 bp, Artificial Sequence
  Nucleic acid encoding heterodimer mil12ail2- il12bgmcsf
atgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggtggaggaggttctggaggcggtggaagtggtggcggaggtagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaagagggcagaggaagtcttctaacatgcggtgacgtggaggagaatcccggccctatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggaagtggcggtggaggctctggaggtggcggaagcgcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 74 - DNA, 2472 bp, Artificial Sequence
  Nucleic acid encoding single stranded protein mil12bil12ail2gmcsf
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggaagtggcggtggaggctctggaggtggcggaagcagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggtggaggaggttctggaggcggtggaagtggtggcggaggtagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggcggagggggttcaggtgggggcgggagcggaggtggcggatcggcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 75 - DNA, 3945 bp, Artificial Sequence
  Nucleic acid encoding single stranded protein mil12bil12ail2dianhs76f8gmcsf
atgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcgtgcagcaagtgggcatgtgttccctgcagggtccgatccggaggcggtggaagtggcggtggaggctctggaggtggcggaagcagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggtggaggaggttctggaggcggtggaagtggtggcggaggtagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggcggagggggttcaggtgggggcgggagcggaggtggcggatcgcaggtgcagctgcaggagagcggccccggcctggtgaagcccagcgagaccctgagcctgacctgcgccgtgagcggctacagcatcagcagcggctactactggggctggatcaggcagccccccggcaagggcctggagtggatcggcagcatctaccacagcggcagcacctactacaaccccagcctgaagagcagggtgaccatcagcgtggacaccagcaagaaccagttcagcctgaagctgagcagcgtgaccgccgccgacaccgccgtgtactactgcgccaggggcaagtggagcaagttcgactactggggccagggcaccctggtgaccgtgagcagcggcggtagcggaggggaaattgtgttgacgcagtctccaggcaccctgtctttgtctccaggggaaagagccaccctctcctgcagggccagtcagagtgttagcatgccgtttttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagatgcgtggtcggccgccgacgttcggccaagggaccaaggtggaaatcaaatcttcaagtagcggttcaagttcctctggatcgtcaagctctggtgaggtgcagctgttggagtctgggggaggcttggtacagcctggggggtccctgagactctcctgtgcagcctctggattcacctttagcctgtttacgatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaagtactcatttgtatctttttgactactggggccagggaaccctggtcaccgtctccagtggcggtagcggagggagcagcgagctgacccaggaccccgccgtgagcgtggccctgggccagaccgtgaggatcacctgccagggcgacagcctgaggagctactacgccagctggtaccagcagaagcccggccaggcccccgtgctggtgatctacggcaagaacaacaggcccagcggcatccccgacaggttcagcggcagcagcagcggcaacaccgccagcctgaccatcaccggcgcccaggccgaggacgaggccgactactactgcaacagcagggacagcagcggcaaccacgtggtgttcggcggcggcaccaagctgaccgtgctgtcaagttcctctggtagttcatctagcggtagttcttcatcgggtgcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaa

SEQ ID NO: 76 - protein, 149 aa, Rattus norvegicus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 77 - protein, 133 aa, Homo sapiens [sequence omitted]

(19) United States
(12) Patent Application Publication
(10) Pub. No.: US 2023/0043257 A1
(43) Pub. Date: Feb. 9, 2023

(54) PHARMACEUTICAL COMPOSITION AND USE THEREOF

(71) Applicant: Jinyu ZHANG, Chongqing (CN)

(72) Inventor: Jinyu ZHANG, Chongqing (CN)

(21) Appl. No.: 17/759,114

(22) PCT Filed: Jan. 20, 2021
(86) PCT No.: PCT/CN2021/072824

§ 371 Date: Jul. 20, 2022

(30) Foreign Application Priority Data
Jan. 21, 2020 (CN) 202010071620.X

(51) Int. Cl. A61K 47/68; A61K 38/20; A61K 38/19; A61K 39/395; A61P 35/00

(52) U.S. Cl. CPC A61K 47/6813; A61K 38/2013; A61K 38/208; A61K 38/193; A61K 47/6851; A61K 39/3955; A61P 35/00

(57) ABSTRACT

A pharmaceutical composition is described, which comprises proteins and an immune checkpoint inhibitor, wherein the proteins comprise a fusion protein, and the fusion protein comprises cytokines IL12, IL2, and GMCSF. A reagent kit is also described, which comprises the pharmaceutical composition. The pharmaceutical composition or the reagent kit may be used in preparing a medicament for treating a tumor.

### TECHNICAL FIELD

The present application relates to the field of biomedicine, and specifically to a pharmaceutical composition and a use thereof.

### BACKGROUND OF THE INVENTION

Tumor is a disease that seriously threatens human health. In recent years, immunotherapy, as a new therapy, has shown great potential in tumor treatment. Cytokines are very important immune signals in vivo, and the cytokine fusion protein technology is another hot direction for tumor immunotherapy today. This method is the fusion of two or more cytokines using genetic engineering techniques based on the fact that these cytokines have the same or related functional activities while each has a different target of action. However, at present, the effect of tumor treatment using the cytokine fusion protein technology is still unsatisfactory, and there is much to be improved.

### SUMMARY OF THE INVENTION

In one aspect, the present application provides a pharmaceutical composition, which includes proteins and an immune checkpoint inhibitor, wherein the proteins include a fusion protein, and the fusion protein includes cytokines IL12, IL2, and GMCSF.

In some embodiments, the immune checkpoint inhibitor includes inhibitors of PD1, PD-L 1 and/or CTLA-4.

In some embodiments, the cytokines are derived from mammals.

In some embodiments, the proteins further include a targeting moiety.

In some embodiments, the targeting moiety can specifically recognize and/or bind to a tumor-associated antigen.

In some embodiments, the tumor-associated antigen is selected from the following group: an EDB domain of fibronectin, an EDA domain of fibronectin, and a necrotic region.

In some embodiments, the targeting moiety includes an antibody or an antigen binding fragment thereof.

In some embodiments, the targeting moiety includes an amino acid sequence as set forth in any one of the following group: SEQ ID NOs. 1-15.

In some embodiments, the proteins are single-stranded proteins.

In some embodiments, the single-stranded protein includes an amino acid sequence as set forth in any one of the following group: SEQ ID NOs. 27-52.

In some embodiments, the proteins are dimers composed of a first polypeptide chain and a second polypeptide chain, and the first polypeptide chain is different from the second polypeptide chain.

In some embodiments, the first polypeptide chain includes IL12a, and the second polypeptide chain includes IL12b.

In some embodiments, the IL2 or a functional fragment thereof is located in the first polypeptide chain or the second polypeptide chain, the GMCSF or a functional fragment thereof is located in the first polypeptide chain or the second polypeptide chain.

In some embodiments, the IL2 or the functional fragment thereof is located in the first polypeptide chain or the second polypeptide chain, the GMCSF or the functional fragment thereof is located in the first polypeptide chain or the second polypeptide chain, and one or more of the targeting moieties are each independently located in the first polypeptide chain or the second polypeptide chain.

In some embodiments, in the first polypeptide chain, the IL12a or the functional fragment thereof and the IL2 or the functional fragment thereof are sequentially included from N-terminus to C-terminus; alternatively, in the first polypeptide chain, the IL2 or the functional fragment thereof and the IL12a or the functional fragment thereof are sequentially included from N-terminus to C-terminus; also alternatively, in the first polypeptide chain, the IL12a or the functional fragment thereof and the GMCSF or the functional fragment thereof are sequentially included from N-terminus to C-terminus.

In some embodiments, in the second polypeptide chain, the IL12b or the functional fragment thereof and the GMCSF or the functional fragment thereof are sequentially included from N-terminus to C-terminus; alternatively, in the second polypeptide chain, the GMCSF or the functional fragment thereof and the IL12b or the functional fragment thereof are sequentially included from N-terminus to C-terminus; also alternatively, in the second polypeptide chain, the IL12b or the functional fragment thereof and the IL2 or the functional fragment thereof are sequentially included from N-terminus to C-terminus.

In some embodiments, in the pharmaceutical composition,

a) the first polypeptide chain includes an amino acid sequence as set forth in SEQ ID NO. 53 and the second polypeptide chain includes an amino acid sequence as set forth in SEQ ID NO. 57;

b) the first polypeptide chain includes an amino acid sequence as set forth in SEQ ID NO. 54 and the second polypeptide chain includes an amino acid sequence as set forth in SEQ ID NO. 57;

c) the first polypeptide chain includes an amino acid sequence as set forth in SEQ ID NO. 53 and the second polypeptide chain includes an amino acid sequence as set forth in SEQ ID NO. 58;

d) the first polypeptide chain includes an amino acid sequence as set forth in SEQ ID NO. 54 and the second polypeptide chain includes an amino acid sequence as set forth in SEQ ID NO. 58;

e) the first polypeptide chain includes an amino acid sequence as set forth in SEQ ID NO. 55 and the second polypeptide chain includes an amino acid sequence as set forth in SEQ ID NO. 59;

f) the first polypeptide chain includes an amino acid sequence as set forth in SEQ ID NO. 56 and the second polypeptide chain includes an amino acid sequence as set forth in SEQ ID NO. 60.

In some embodiments, in the first polypeptide chain, the targeting moiety, the IL12a or the functional fragment thereof, the IL2 or the functional fragment thereof and the GMCSF or the functional fragment thereof are sequentially included from N-terminus to C-terminus; alternatively, in the first polypeptide chain, the IL2 or the functional fragment thereof, the IL12a or the functional fragment thereof and the GMCSF or the functional fragment thereof are sequentially included from N-terminus to C-terminus.

In some embodiments, in the second polypeptide chain, the IL12b or the functional fragment thereof and the targeting moiety are sequentially included from N-terminus to C-terminus.

In some embodiments, in the pharmaceutical composition,

1) the first polypeptide chain includes an amino acid sequence as set forth in SEQ ID NO. 66 and the second polypeptide chain includes an amino acid sequence as set forth in SEQ ID NO. 61;

2) the first polypeptide chain includes an amino acid sequence as set forth in SEQ ID NO. 66 and the second polypeptide chain includes an amino acid sequence as set forth in SEQ ID NO. 62;

3) the first polypeptide chain includes an amino acid sequence as set forth in SEQ ID NO. 66 and the second polypeptide chain includes an amino acid sequence as set forth in SEQ ID NO. 63;

4) the first polypeptide chain includes an amino acid sequence as set forth in SEQ ID NO. 67 and the second polypeptide chain includes an amino acid sequence as set forth in SEQ ID NO. 61;

5) the first polypeptide chain includes an amino acid sequence as set forth in SEQ ID NO. 68 and the second polypeptide chain includes an amino acid sequence as set forth in SEQ ID NO. 62;

6) the first polypeptide chain includes an amino acid sequence as set forth in SEQ ID NO. 69 and the second polypeptide chain includes an amino acid sequence as set forth in SEQ ID NO. 63;

7) the first polypeptide chain includes an amino acid sequence as set forth in SEQ ID NO. 70 and the second polypeptide chain includes an amino acid sequence as set forth in SEQ ID NO. 64;

8) the first polypeptide chain includes an amino acid sequence as set forth in SEQ ID NO. 71 and the second polypeptide chain includes an amino acid sequence as set forth in SEQ ID NO. 64;

9) the first polypeptide chain includes an amino acid sequence as set forth in SEQ ID NO. 72 and the second polypeptide chain includes an amino acid sequence as set forth in SEQ ID NO. 65.

In another aspect, the present application provides a reagent kit including the pharmaceutical composition.

In another aspect, the present application further provides uses of the pharmaceutical composition or the reagent kit in preparing a medicament for treating a tumor.

In some embodiments, the tumor includes a solid tumor.

In some embodiments, the tumor includes melanoma.

In some embodiments, the tumor includes breast cancer.

In some embodiments, the tumor includes lung cancer.

In some embodiments, the pharmaceutical composition or the reagent kit of the present invention, which is used for treating a tumor.

In another aspect, the present application further provides a method for treating a tumor, which includes administering to a subject in need thereof the pharmaceutical composition or the reagent kit.

In some embodiments, the administration includes administering the proteins first, and then administering the immune checkpoint inhibitor.

In some embodiments, the administration includes intratumoral injection, intravenous injection, or subcutaneous injection.

In some embodiments, the tumor includes a solid tumor.

In some embodiments, the tumor includes melanoma.

In some embodiments, the tumor includes breast cancer.

In some embodiments, the tumor includes lung cancer.

Other aspects and advantages of the present application can be readily perceived by those skilled in the art from the following detailed description. In the following detailed description, only exemplary embodiments of the present application are shown and described. As will be recognized by those skilled in the art, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWING

The specific features of the invention involved in the present application are as shown in the appended claims. The characteristics and advantages of the invention involved in the present application can be better understood by referring to the exemplary embodiments described in detail below and the accompanying drawings. A brief description of the drawings is as below:

FIGS. 1A-1C show the effect on the expression of PD1 in T cells after inducing the expression of mIL12aIL2-IL12bGMCSF heterodimer.

FIG. 2 shows the growth of melanoma in mice after treatment with mIL12bIL12aIL2GMCSF and the combination of mIL12bIL12aIL2GMCSF and PD1 antibody according to the present application, respectively.

FIG. 3 shows the growth of melanoma in mice after treatment with mIL12bIL12aIL2DiaNHS76F8GMCSF and the combination of mIL12bIL12aIL2DiaNHS76F8GMCSF and PD1 antibody according to the present application, respectively.

FIG. 4 shows the growth of melanoma in mice after treatment with mIL12aIL2-IL12bGMCSF and the combination of mIL12aIL2-IL12bGMCSF and PD1 antibody according to the present application, respectively.

FIG. 5 shows the growth of breast cancer in mice after treatment with mIL12bIL12aIL2DiaNHS76F8GMCSF and the combination of mIL12bIL12aIL2DiaNHS76F8GMCSF and PD1 antibody according to the present application, respectively.

FIG. 6 shows the growth of lung cancer in mice after treatment with mIL12bIL12aIL2DiaNHS76F8GMCSF and the combination of mIL12bIL12aIL2DiaNHS76F8GMCSF and PD1 antibody according to the present application, respectively.

### DETAILED DESCRIPTION

The implementation of the present application will be illustrated below by specific examples, and other advantages and effects of the present application will be easily known by those familiar with the art from the contents disclosed in the specification.

Definition of Terms

In the present application, the term “pharmaceutical composition” generally refers to a composition suitable for administering to a patient. The pharmaceutical composition of the present application includes proteins and an immune checkpoint inhibitor, wherein the proteins include a fusion protein, and the fusion protein includes cytokines IL2, IL2, and GMCSF. In some embodiments, the pharmaceutical composition may also include one or more (pharmaceutically effective) carriers, stabilizers, excipients, diluents, solubilizers, surfactants, emulsifiers, preservatives and/or adjuvants and other suitable preparations. For example, the acceptable ingredients for the composition may be non-toxic to the recipient at the dosage and concentration used. The pharmaceutical composition of the present application includes, but not limited to, liquid, frozen and freeze-dried compositions.

In the present application, the term “immune checkpoint inhibitor” generally refers to a molecule that, in whole or in part, reduces, inhibits, interferes with, or modulates one or more checkpoint proteins. Checkpoint proteins modulate the activation and function of T cells. Various checkpoint proteins are known, e.g., CTLA-4 and its ligands CD80 and CD86; as well as PD1 and its ligands PD-L1 and PD-L2 (Pardoll, Nature Reviews Cancer 12:252-264, 2012). These proteins are responsible for the co-stimulatory or inhibitory interactions of T cell responses. Immune checkpoint proteins regulate and maintain self-tolerance as well as the duration and amplitude of physiological immune responses. The immune checkpoint inhibitor includes an antibody or is derived from an antibody. For example, in the present application, the immune checkpoint inhibitor can include inhibitors of PD1, PD-L1 and/or CTLA-4.

In the present application, the term “protein” can be considered to belong to “cytokine fusion protein”, which generally refers to a fusion protein that can be obtained by fusing two or more cytokines together through genetic recombination techniques. It has the unique biological activities of its constituent factors or significantly enhances some of their activities, and it may also exert complex biological functions that are not available in simple combinations of single cytokines through the complementary and synergistic effects of biological activities, and some new structures and biological functions may even be generated.

In the present application, the terms “IL12”, “IL12a”, IL12b″, “IL2”, and “GMCSF” may be considered to belong to “cytokines”. The “cytokines” generally refer to a class of small molecule proteins with a wide range of biological activities that are synthesized and secreted from immune cells (e.g., monocytes, macrophages, T cells, B cells, NK cells, etc.) and some non-immune cells (e.g., endothelial cells, epidermal cells, fibroblasts, etc.) upon stimulation. The cytokines have important regulatory effects on cell-cell interactions, cell growth and differentiation. In the present application, the cytokines may be selected from one or more of the following group: interleukins (ILs) and colony stimulating factors (CSFs). The interleukins generally refer to cytokines produced from lymphocytes, monocytes or other non-mononuclear cells. In the present application, the interleukins may be selected from one or more of the following group: IL12, IL2. In the present application, the colony stimulating factors generally refer to cytokines that can stimulate different hematopoietic stem cells to form cell colonies in semi-solid medium. In the present application, the colony stimulating factors may be granulocyte macrophage colony stimulating factors (GMCSFs).

In the present application, the term “L12” generally refers to interleukin-12, which can play important regulatory roles in the processes of cell-cell interactions, immune regulation, hematopoiesis, and inflammation. The molecule of IL12 is usually a heterodimer, which usually consists of two subunits, a p40 subunit (40 kd) and a p35 subunit (35 kd), which are linked together by a disulfide bond. In the present application, the IL12 containing the p35 subunit (35 kd) can be represented as IL12a, and the IL12 containing the p40 subunit (40 kd) can be represented as IL12b. For example, in the mouse-derived IL12 (mIL12), the p35 subunit can include an amino acid sequence as set forth in SEQ ID NO. 16 and the p40 subunit can include an amino acid sequence as set forth in SEQ ID NO. 17. Further for example, in the human-derived IL12 (hIL12), the p35 subunit can include an amino acid sequence as set forth in SEQ ID NO. 18 and the p40 subunit can include an amino acid sequence as set forth in SEQ ID NO. 19.

In the present application, the term “IL2” generally refers to interleukin-2, which plays important regulatory roles in the processes of cell-cell interactions, immune regulation, hematopoiesis, and inflammation. For example, the mouse-derived IL2 (mIL2) can include an amino acid sequence as set forth in SEQ ID NO. 76, and the human-derived IL12 (hIL12) can include an amino acid sequence as set forth in SEQ ID NO. 77.

In the present application, the term “GMCSF” generally refers to a granulocyte macrophage colony stimulating factor. The GMCSF can carry 4 α-helix bundle structures. For example, a mouse-derived GMCSF (mGMCSF) can include an amino acid sequence as set forth in SEQ ID NO. 20. Further for example, a human-derived GMCSF (hGMCSF) can include an amino acid sequence as set forth in SEQ ID NO. 21.

In the present application, the term “antibody” generally refers to an immunoglobulin or a fragment or derivative thereof, encompassing any polypeptide that includes an antigen binding site, no matter whether it is produced in vitro o in vivo. The term includes, but is not limited to, polyclonal, monoclonal, mono-specific, multi-specific, non-specific, humanized, single-chain, chimeric, synthetic, recombinant, hybrid, mutated and grafted antibodies. Unless otherwise modified by a term “complete”, as in “complete antibody”, for the purposes of the present invention, the term “antibody” also includes antibody fragments, such as Fab, F(ab′)₂, Fv, scFv, Fd, dAb and other antibody fragments that retain the antigen binding functions. In general, such fragments should include antigen-binding domains.

The basic 4-chain antibody unit is a heterotetrameric glycoprotein composed of two identical light (L) chains and two identical heavy (H) chains. The IgM antibody is composed of 5 basic heterotetrameric units and another polypeptide called J chain, and contains 10 antigen-binding sites; while the IgA antibody includes 2-5 basic 4-chain units that can be polymerized with the J chain to form a multivalent combination. In terms of IgG, the 4-chain unit is generally about 150,000 Daltons. Each L chain is linked to the H chain through a covalent disulfide bond, while two H chains are linked to each other through one or more disulfide bonds depending on the isotype of the H chain. Each H and L chain also has regularly spaced intra-chain disulfide bridges. Each H chain has a variable domain (VH) at the N-terminus, which is followed by three constant domains (CH) for each of α and γ chains, and followed by four CH domains for μ and ε isotypes. Each L chain has a variable domain (VL) at the N-terminus, and has a constant domain at the other terminus. VL corresponds to VH, and CL corresponds to the first constant domain (CH1) of the heavy chain. Specific amino acid residues are considered to form an interface between the light chain and heavy chain variable domains. VH is paired with VL to form a single antigen-binding site. For the structures and properties of different kinds of antibodies, see for example Basic and Clinical Immunology, 8th Edition, Daniel P. Sties, Abba I. Terr and Tristram G. Parsolw (eds), Appleton & Lange, Norwalk, Conn., 1994, Page 71 and Chapter 6. L chains from any vertebrate species can be classified into one of two distinct types based on the amino acid sequence of their constant domains, called kappa and lambda. Depending on the amino acid sequence of the constant domain of the heavy chain (CH), immunoglobulin can be classified into different types or isotypes. There are five types of immunoglobulin: IgA, IgD, IgE, IgG and IgM, which have heavy chains named α, δ, ε, γ and μ, respectively. Based on the relatively small differences in terms of CH sequence and function, the γ and α types are further divided into sub-types. For example, human expresses the following subtypes: IgG1, IgG2A, IgG2B, IgG3, IgG4, IgA1 and IgK1.

In the present application, the term “targeting moiety” generally refers to a class of parts that act against certain specific tissues and cells. For example, the targeting moiety can specifically target a tumor-associated antigen. In the present application, the targeting moiety includes an antibody or an antigen binding fragment thereof.

In the present application, the term “specifically recognize and/or bind to” generally refers to measurable and reproducible interactions, such as the binding between targets and antibodies. For example, an antibody specifically binding to a target (which may be an epitope) is an antibody that binds to the target with greater affinity, avidity, easier, and/or for a longer duration than it binds to other targets. In some embodiments, the antibody specifically binds to the epitope on the proteins, and the epitope is conservative among proteins of different species. In another embodiment, specific binding can include, but does not require exclusive binding.

In the present application, the term “tumor-associated antigen (TAA)” generally refers to antigenic molecules present on tumor cells or normal cells. The tumor-associated antigen can include: embryonic protein, glycoprotein antigen and squamous cell antigen. The tumor-associated antigen may be selected from the following group: an EDB domain of fibronectin, an EDA domain of fibronectin, and a necrotic region.

In the present application, the term “antigen-binding fragment” generally refers to fragments with antigen-binding activities. In the present application, the antigen-binding fragment may be selected from the following group: Fab, Fab′, F(ab′)₂, F(ab)₂, dAb, an isolated complementarity-determining region (CDR), Fv and scFv.

In the present application, the term “single-stranded protein” generally refers to a polypeptide with a primary structure consisting of an uninterrupted sequence of consecutive amino acid residues. For example, in the present application, the single-stranded protein can include an amino acid sequence as set forth in any one of the following group: SEQ ID NOs. 27-52.

In the present application, the term “dimer”generally refers to a polymer complex formed from two monomer units that are usually non-covalently bonded. Each monomer unit may be a macromolecule, e.g., a polypeptide chain or a polynucleotide. For example, in the present application, the proteins may be dimers composed of a first polypeptide chain and a second polypeptide chain.

In the present application, the term “polypeptide chain” generally refers to a macromolecule including two or more covalently linked peptides. The peptides within a polypeptide chain can be linked to each other through peptide bonds. Each polypeptide chain can include one N-terminus or amino terminus and one C-terminus or carboxyl terminus.

In the present application, the term “functional fragment” generally refers to a fragment that retains a certain specific function. For example, the IL12a functional fragment refers to a fragment that retains the function of IL12a. For example, the IL12a functional fragment may be IL12a, fragment (GenBank: AIC49052.1). For example, the IL12b functional fragment may be IL12b, fragment (GenBank: AIC54621.1).

In the present application, the term “reagent kit” generally refers to a packaged product containing the pharmaceutical composition of the present application. The reagent kit can include a box or container containing the components of the reagent kit. The box or container is attached with a label or a FDA-approved treatment regimen. The box or container contains the components of the pharmaceutical composition of the present application, for example, the components can be contained in a plastic, polyethylene, polypropylene, ethylene or propylene container. The container can be a capped tube or bottle. In addition, the reagent kit can also contain instructions for administering the pharmaceutical composition of the present application.

In the present application, the term “tumor” generally refers to a neoplasm or a solid lesion formed by abnormal cell growth. In the present application, the tumor may be a solid tumor or a blood tumor. For example, the tumor may include melanoma.

In the present application, the term “subject” generally refers to human or non-human animals, including, but not limited to, cat, dog, horse, pig, cow, sheep, rabbit, mouse, rat, or monkey.

In the present application, the term “administration” generally refers to a method of giving a subject (e.g., a patient) a certain dose of liquid preparations or drugs. Administration can be conducted by any suitable ways, including parenterally, intrapulmonarily and intranasally, as well as (if required by topical treatment) intralesional administration. Parenteral infusion includes, e.g., intramuscular, intravenous, intra-artery, intraperitoneal or subcutaneous administration. Administration may be done through any suitable routes, for example by injection (such as intravenous or subcutaneous injection), depending in part on whether the administration is transient or long-term. Various dosing schedules are contemplated herein, including, but not limited to, a single administration or multiple administrations at various time points, bolus administration, and pulse infusion. For example, in the present application, the administration can be intratumoral injection. The “intratumoral injection” generally refers to the injection of a certain dose of liquid preparations or drugs into the tumor. In some cases, the administration can also be intravenous injection or subcutaneous injection.

In the present application, the term “include” generally refers to the inclusion of explicitly specified features, but not excluding other elements.

In the present application, the term “about” generally refers to varying in a range of 0.5%-10% above or below a specified value, for example, varying in a range of 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below a specified value.

### DETAILED DESCRIPTION OF THE INVENTION

Pharmaceutical Composition and Reagent Kit

In one aspect, the present application provides a pharmaceutical composition, which includes proteins and an immune checkpoint inhibitor, wherein the proteins include a fusion protein, and the fusion protein includes cytokines IL12, IL2, and GMCSF.

In the present application, the immune checkpoint inhibitor can include inhibitors of PD1, PD-L1 and/or CTLA-4. For example, the immune checkpoint inhibitor may be an antibody of PD1, PD-L1 and/or CTLA-4.

In the present application, the cytokines may be derived from mammals. For example, in some embodiments, the mammals may be human or mice. For example, the amino acid sequence of a mouse-derived IL12a (represented as mIL12a) may be as set forth in SEQ ID NO. 16, the amino acid sequence of a mouse-derived IL12b (represented as mIL12b) may be as set forth in SEQ ID NO. 17, and the amino acid sequence of a mouse-derived GMCSF (represented as mGMCSF) may be as set forth in SEQ ID NO. 20. Further for example, the amino acid sequence of a human-derived IL12a (represented as hIL12a) may be as set forth in SEQ ID NO. 18, the amino acid sequence of a human-derived IL12b (represented as hIL12b) may be as set forth in SEQ ID NO. 19, and the amino acid sequence of a human-derived GMCSF (represented as hGMCSF) may be as set forth in SEQ ID NO. 21. Further for example, the amino acid sequence of a mouse-derived IL2 may be as set forth in SEQ ID NO. 76, and the amino acid sequence of a human-derived IL2 may be as set forth in SEQ ID NO. 77.

In the present application, the proteins may further include a targeting moiety. The number of the targeting moiety may be one or more. The targeting moieties may be the same, and may also be different. The targeting moiety can specifically recognize and/or bind to a tumor-associated antigen. The tumor-associated antigen may be selected from the following group: an EDB domain of fibronectin, an EDA domain of fibronectin, and a necrotic region.

In the present application, the targeting moiety can include an antibody or an antigen binding fragment thereof. The antigen-binding fragment may be selected from the following group: Fab, Fab′, F(ab′)₂, F(ab)₂, dAb, an isolated complementarity-determining region (CDR), Fv and scFv. In some embodiments, the antigen-binding fragment may be scFv.

In the present application, the targeting moiety can include an amino acid sequence as set forth in any one of the following group: SEQ ID NOs. 1-15.

For example, the targeting moiety of the proteins may be selected from the following group: L19V_{L} (its amino acid sequence may be as set forth in SEQ ID NO. 10), L19V_{H} (its amino acid sequence may be as set forth in SEQ ID NO. 11), F8V_{L} (its amino acid sequence may be as set forth in SEQ ID NO. 12), F8V_{H} (its amino acid sequence may be as set forth in SEQ ID NO. 13), NHS76V_{L} (its amino acid sequence may be as set forth in SEQ ID NO. 14), and NHS76V_{H} (its amino acid sequence may be as set forth in SEQ ID NO. 15).

In the present application, the cytokines can be linked to each other or to the targeting moieties through a linker. The linker may be a linker peptide. In some embodiments, the linker may contain a thrombin cleavage site.

In the present application, the linker can include an amino acid sequence as set forth in any one of the following group: SEQ ID NOs. 22-26.

For example, the cytokines can be linked to each other through the linker. In the present application, the IL12a, IL12b, IL2 and GMCSF can be linked to each other through the linker peptide. For example, the linker peptide can include an amino acid sequence as set forth in any one of SEQ ID NOs. 22 and 24.

For example, the cytokines can be linked to the targeting moieties through the linker. In the present application, the targeting moieties can be linked to IL12a, IL12b, IL2 and GMCSF through the linker peptide. For example, the linker peptide can include an amino acid sequence as set forth in any one of SEQ ID NOs. 22-26.

In the present application, the proteins may be a single-stranded protein, and the single-stranded protein can include an amino acid sequence as set forth in any one of the following group: SEQ ID NOs. 27-52.

For example, the structure of the single-stranded protein can be that, the C-terminus of mIL12b is fused to the N-terminus of mIL12a, the C-terminus of mIL12a is fused to the N-terminus of mIL2, and the C-terminus of mIL2 is fused to the N-terminus of mGMCSF, thus forming an mIL12b-mIL12a-mIL2-mGMCSF protein molecule (its amino acid sequence can be as set forth in SEQ ID NO. 27), which can be referred to as mIL12bIL12aIL2GMCSF protein.

For example, the structure of the single-stranded protein can be that, the C-terminus of mIL12b is fused to the N-terminus of mIL12a, the C-terminus of mIL12a is fused to the N-terminus of mGMCSF, and the C-terminus of mGMCSF is fused to the N-terminus of mIL2, thus forming an mIL12b-mIL12a-mGMCSF-mIL2 protein molecule (its amino acid sequence can be as set forth in SEQ ID NO. 28), which can be referred to as mIL12bIL12aGMCSFIL2 protein.

For example, the structure of the single-stranded protein can be that, the C-terminus of hIL12b is fused to the N-terminus of hIL12a, the C-terminus of hIL12a is fused to the N-terminus of hIL2, and the C-terminus of hIL2 is fused to the N-terminus of hGMCSF, thus forming an hIL12b-hIL12a-hIL2-hGMCSF protein molecule (its amino acid sequence can be as set forth in SEQ ID NO. 29), which can be referred to as hIL12bIL12aIL2GMCSF protein.

For example, the structure of the single-stranded protein can be that, the C-terminus of mIL12b is fused to the N-terminus of mIL12a, the C-terminus of mIL12a is fused to the N-terminus of mL19V_{H}, the C-terminus of mL19V_{H} is fused to the N-terminus of mL19V_{L}, the C-terminus of mL19V_{L} is fused to the N-terminus of mL19V_{H}, the C-terminus of mL19V_{H} is fused to the N-terminus of mL19V_{L}, the C-terminus of mL19V_{L} is fused to the N-terminus of mIL2, and the C-terminus of mIL2 is fused to the N-terminus of mGMCSF, thus forming an mIL12b-mIL12a-mL19V_{H}-mL19V_{L}-mL19V_{H}-mL19V_{L}-mIL2-mGMCSF protein molecule (its amino acid sequence can be as set forth in SEQ ID NO. 30), which can be referred to as mIL12bIL12aDiaL19IL2GMCSF protein.

For example, the structure of the single-stranded protein can be that, the C-terminus of mIL12b is fused to the N-terminus of mIL12a, the C-terminus of mIL12a is fused to the N-terminus of mF8V_{H}, the C-terminus of mF8V_{H} is fused to the N-terminus of mF8V_{L}, the C-terminus of mF8V_{L} is fused to the N-terminus of mF8V_{H}, the C-terminus of mF8V_{H} is fused to the N-terminus of mF8V_{L}, the C-terminus of mF8V_{L} is fused to the N-terminus of mIL2, and the C-terminus of mIL2 is fused to the N-terminus of mGMCSF, thus forming an mIL12b-mIL12a-mF8V_{H}-mF8V_{L}-mF8V_{H}-mF8V_{L}-mIL2-mGMCSF protein molecule (its amino acid sequence can be as set forth in SEQ ID NO. 31), which can be referred to as mIL12bIL12aDiaF8IL2GMCSF protein.

For example, the structure of the single-stranded protein can be that, the C-terminus of mIL12b is fused to the N-terminus of mIL12a, the C-terminus of mIL12a is fused to the N-terminus of mNHS76V_{H}, the C-terminus of mNHS76V_{H} is fused to the N-terminus of mNHS76V_{L}, the C-terminus of mNHS76V_{L} is fused to the N-terminus of mNHS76V_{H}, the C-terminus of mNHS76V_{H} is fused to the N-terminus of mNHS76V_{L}, the C-terminus of mNHS76V_{L} is fused to the N-terminus of mIL2, and the C-terminus of mIL2 is fused to the N-terminus of mGMCSF, thus forming an mIL12b-mIL12a-mNHS76V_{H}-mNHS76V_{L}-mNHS76V_{H}-mNHS76V_{L}-mIL2-mGMCSF protein molecule (its amino acid sequence can be as set forth in SEQ ID NO. 32), which can be referred to as mIL12bIL12aDiaNHS76IL2GMCSF protein.

For example, the structure of the single-stranded protein can be that, the C-terminus of mIL12b is fused to the N-terminus of mIL12a, the C-terminus of mIL12a is fused to the N-terminus of mNHS76V_{H}, the C-terminus of mNHS76V_{H} is fused to the N-terminus of mF8V_{L}, the C-terminus of mF8V_{L} is fused to the N-terminus of mF8V_{H}, the C-terminus of mF8V_{H} is fused to the N-terminus of mNHS76V_{L}, the C-terminus of mNHS76V_{L} is fused to the N-terminus of mIL2, and the C-terminus of mIL2 is fused to the N-terminus of mGMCSF, thus forming an mIL12b-mIL12a-mNHS76V_{H}-mF8V_{L}-mF8V_{H}-mNHS76V_{L}-mIL2-mGMCSF protein molecule (its amino acid sequence can be as set forth in SEQ ID NO. 33), which can be referred to as mIL12bIL12aDiaNHS76F8IL2GMCSF protein.

For example, the structure of the single-stranded protein can be that, the C-terminus of mIL12b is fused to the N-terminus of mIL12a, the C-terminus of mIL12a is fused to the N-terminus of mNHS76V_{H}, the C-terminus of mNHS76V_{H} is fused to the N-terminus of mL19V_{L}, the C-terminus of mL19V_{L} is fused to the N-terminus of mL19V_{H}, the C-terminus of mL19V_{H} is fused to the N-terminus of mNHS76V_{L}, the C-terminus of mNHS76V_{L} is fused to the N-terminus of mIL2, and the C-terminus of mIL2 is fused to the N-terminus of mGMCSF, thus forming an mIL12b-mIL12a-mNHS76V_{H}-mL19V_{L}-mL19V_{H}-mNHS76V_{L}-mIL2-mGMCSF protein molecule (its amino acid sequence can be as set forth in SEQ ID NO. 34), which can be referred to as mIL12bIL12aDiaNHS76L19IL2GMCSF protein.

For example, the structure of the single-stranded protein can be that, the C-terminus of mIL12b is fused to the N-terminus of mIL12a, the C-terminus of mIL12a is fused to the N-terminus of mF8V_{H}, the C-terminus of mF8V_{H} is fused to the N-terminus of mNHS76V_{L}, the C-terminus of mNHS76V_{L} is fused to the N-terminus of mNHS76V_{H}, the C-terminus of mNHS76V_{H} is fused to the N-terminus of mF8V_{L}, the C-terminus of mF8V_{L} is fused to the N-terminus of mIL2, and the C-terminus of mIL2 is fused to the N-terminus of mGMCSF, thus forming an mIL12b-mIL12a-mF8V_{H}-mNHS76V_{L}-mNHS76V_{H}-mF8V_{L}-mIL2-mGMCSF protein molecule (its amino acid sequence can be as set forth in SEQ ID NO. 35), which can be referred to as mIL12bIL12aDiaF8NHS76IL2GMCSF protein.

For example, the structure of the single-stranded protein can be that, the C-terminus of mIL12b is fused to the N-terminus of mIL12a, the C-terminus of mIL12a is fused to the N-terminus of mF8V_{H}, the C-terminus of mF8V_{H} is fused to the N-terminus of mL19V_{L}, the C-terminus of mL19V_{L} is fused to the N-terminus of mL19V_{H}, the C-terminus of mL19V_{H} is fused to the N-terminus of mF8V_{L}, the C-terminus of mF8V_{L} is fused to the N-terminus of mIL2, and the C-terminus of mIL2 is fused to the N-terminus of mGMCSF, thus forming an mIL12b-mIL12a-mF8V_{H}-mL19V_{L}-mL19V_{H}-mF8V_{L}-mIL2-mGMCSF protein molecule (its amino acid sequence can be as set forth in SEQ ID NO. 36), which can be referred to as mIL12bIL12aDiaF8L19IL2GMCSF protein.

For example, the structure of the single-stranded protein can be that, the C-terminus of mIL12b is fused to the N-terminus of mIL12a, the C-terminus of mIL12a is fused to the N-terminus of mL19V_{H}, the C-terminus of mL19V_{H} is fused to the N-terminus of mNHS76V_{L}, the C-terminus of mNHS76V_{L} is fused to the N-terminus of mNHS76V_{H}, the C-terminus of mNHS76V_{H} is fused to the N-terminus of mL19V_{L}, the C-terminus of mL19V_{L} is fused to the N-terminus of mIL2, and the C-terminus of mIL2 is fused to the N-terminus of GMCSF, thus forming an mIL12b-mIL12a-mL19V_{H}-mNHS76V_{L}-mNHS76V_{H}-mL19V_{L}-mIL2-mGMCSF protein molecule (its amino acid sequence can be as set forth in SEQ ID NO. 37), which can be referred to as mIL12bIL12aDiaL19NHS76IL2GMCSF protein.

For example, the structure of the single-stranded protein can be that, the C-terminus of mIL12b is fused to the N-terminus of mIL12a, the C-terminus of mIL12a is fused to the N-terminus of mL19V_{H}, the C-terminus of mL19V_{H} is fused to the N-terminus of mF8V_{L}, the C-terminus of mF8V_{L} is fused to the N-terminus of mF8V_{H}, the C-terminus of mF8V_{H} is fused to the N-terminus of mL19V_{L}, the C-terminus of mL19V_{L} is fused to the N-terminus of mIL2, and the C-terminus of mIL2 is fused to the N-terminus of mGMCSF, thus forming an mIL12b-mIL12a-mL19V_{H}-mF8V_{L}-mF8V_{H}-mL19V_{L}-mIL2-mGMCSF protein molecule (its amino acid sequence can be as set forth in SEQ ID NO. 38), which can be referred to as mIL12bIL12aDiaL19F8IL2GMCSF protein.

For example, the structure of the single-stranded protein can be that, the C-terminus of mIL12b is fused to the N-terminus of mIL12a, the C-terminus of mIL12a is fused to the N-terminus of mIL2, the C-terminus of mIL2 is fused to the N-terminus of mNHS76V_{H}, the C-terminus of mNHS76V_{H} is fused to the N-terminus of mF8V_{L}, the C-terminus of mF8V_{L} is fused to the N-terminus of mF8V_{H}, the C-terminus of mF8V_{H} is fused to the N-terminus of mNHS76V_{L}, the C-terminus of mNHS76V_{L} is fused to the N-terminus of mGMCSF, thus forming an mIL12b-mIL12a-mIL2-mNHS76V_{H}-mF8V_{L}-mF8V_{H}-mNHS76V_{L}-mGMCSF protein molecule (its amino acid sequence can be as set forth in SEQ ID NO. 39), which can be referred to as mIL12bIL12aIL2DiaNHS76F8GMCSF protein.

For example, the structure of the single-stranded protein can be that, the C-terminus of mIL12b is fused to the N-terminus of mIL12a, the C-terminus of mIL12a is fused to the N-terminus of mIL2, the C-terminus of mIL2 is fused to the N-terminus of mF8V_{H}, the C-terminus of mF8V_{H} is fused to the N-terminus of mF8V_{L}, the C-terminus of mF8V_{L} is fused to the N-terminus of mF8V_{H}, the C-terminus of mF8V_{H} is fused to the N-terminus of mF8V_{L}, the C-terminus of mF8V_{L} is fused to the N-terminus of mGMCSF, thus forming an mIL12b-mIL12a-mIL2-mF8V_{H}-mF8V_{L}-mF8V_{H}-mF8V_{L}-mGMCSF protein molecule (its amino acid sequence can be as set forth in SEQ ID NO. 40), which can be referred to as mIL12bIL12aIL2DiaF8GMCSF protein.

For example, the structure of the single-stranded protein can be that, the C-terminus of mIL12b is fused to the N-terminus of mIL12a, the C-terminus of mIL12a is fused to the N-terminus of mIL2, the C-terminus of mIL2 is fused to the N-terminus of mGMCSF, the C-terminus of mGMCSF is fused to the N-terminus of mNHS76V_{H}, the C-terminus of mNHS76V_{H} is fused to the N-terminus of mF8V_{L}, the C-terminus of mF8V_{L} is fused to the N-terminus of mF8V_{H}, the C-terminus of mF8V_{H} is fused to the N-terminus of mNHS76V_{L}, thus forming an mIL12b-mIL12a-mIL2-mGMCSF-mNHS76V_{H}-mF8V_{L}-mF8V_{H}-mNHS76V_{L} protein molecule (its amino acid sequence can be as set forth in SEQ ID NO. 41), which can be referred to as mIL12bIL12aIL2GMCSFDiaNHS76F8 protein.

For example, the structure of the single-stranded protein can be that, the C-terminus of mIL12b is fused to the N-terminus of mIL12a, the C-terminus of mIL12a is fused to the N-terminus of mIL2, the C-terminus of mIL2 is fused to the N-terminus of mGMCSF, the C-terminus of mGMCSF is fused to the N-terminus of mF8V_{H}, the C-terminus of mF8V_{H} is fused to the N-terminus of mF8V_{L}, the C-terminus of mF8V_{L} is fused to the N-terminus of mF8V_{H}, and the C-terminus of mF8V_{H} is fused to the N-terminus of mF8V_{L}, thus forming an mIL12b-mIL12a-mIL2-mGMCSF-mF8V_{H}-mF8V_{L}-mF8V_{H}-mF8V_{L} protein molecule (its amino acid sequence can be as set forth in SEQ ID NO. 42), which can be referred to as mIL12bIL12aIL2GMCSFDiaF8 protein.

For example, the structure of the single-stranded protein can be that, the C-terminus of hIL12b is fused to the N-terminus of hIL12a, the C-terminus of hIL12a is fused to the N-terminus of hL19V_{H}, the C-terminus of hL19V_{H} is fused to the N-terminus of hL19V_{L}, the C-terminus of hL19V_{L} is fused to the N-terminus of hL19V_{H}, the C-terminus of hL19V_{H} is fused to the N-terminus of hL19V_{L}, the C-terminus of hL19V_{L} is fused to the N-terminus of hIL2, and the C-terminus of hIL2 is fused to the N-terminus of hGMCSF, thus forming an hIL12b-hIL12a-hL19V_{H}-hL19V_{L}-hL19V_{H}-hL19V_{L}-hIL2-hGMCSF protein molecule (its amino acid sequence can be as set forth in SEQ ID NO. 43), which can be referred to as hIL12bIL12aDiaL19IL2GMCSF protein.

For example, the structure of the single-stranded protein can be that, the C-terminus of hIL12b is fused to the N-terminus of hIL12a, the C-terminus of hIL12a is fused to the N-terminus of hNHS76V_{H}, the C-terminus of hNHS76V_{H} is fused to the N-terminus of hNHS76V_{L}, the C-terminus of hNHS76V_{L} is fused to the N-terminus of hNHS76V_{H}, the C-terminus of hNHS76V_{H} is fused to the N-terminus of hNHS76V_{L}, the C-terminus of hNHS76V_{L} is fused to the N-terminus of hIL2, and the C-terminus of hIL2 is fused to the N-terminus of hGMCSF, thus forming an hIL12b-hIL12a-hNHS76V_{H}-hNHS76V_{L}-hNHS76V_{H}-hNHS76V_{L}-hIL2-hGMCSF protein molecule (its amino acid sequence can be as set forth in SEQ ID NO. 44), which can be referred to as hIL12bIL12aDiaNHS76IL2GMCSF protein.

For example, the structure of the single-stranded protein can be that, the C-terminus of hIL12b is fused to the N-terminus of hIL12a, the C-terminus of hIL12a is fused to the N-terminus of hNHS76V_{H}, the C-terminus of hNHS76V_{H} is fused to the N-terminus of hF8V_{L}, the C-terminus of hF8V_{L} is fused to the N-terminus of hF8V_{H}, the C-terminus of hF8V_{H} is fused to the N-terminus of hNHS76V_{L}, the C-terminus of hNHS76V_{L} is fused to the N-terminus of hIL2, and the C-terminus of hIL2 is fused to the N-terminus of hGMCSF, thus forming an hIL12b-hIL12a-hNHS76V_{H}-hF8V_{L}-hF8V_{H}-hNHS76V_{L}-hIL2-hGMCSF protein molecule (its amino acid sequence can be as set forth in SEQ ID NO. 45), which can be referred to as hIL12bIL12aDiaNHS76F8IL2GMCSF protein.

For example, the structure of the single-stranded protein can be that, the C-terminus of hIL12b is fused to the N-terminus of hIL12a, the C-terminus of hIL12a is fused to the N-terminus of hIL2, the C-terminus of hIL2 is fused to the N-terminus of hNHS76V_{H}, the C-terminus of hNHS76V_{H} is fused to the N-terminus of hF8V_{L}, the C-terminus of hF8V_{L} is fused to the N-terminus of hF8V_{H}, the C-terminus of hF8V_{H} is fused to the N-terminus of hNHS76V_{L}, the C-terminus of hNHS76V_{L} is fused to the N-terminus of hGMCSF, thus forming an hIL12b-hIL12a-hIL2-hNHS76V_{H}-hF8V_{L}-hF8V_{H}-hNHS76V_{L}-hGMCSF protein molecule (its amino acid sequence can be as set forth in SEQ ID NO. 46), which can be referred to as hIL12bIL12aIL2DiaNHS76F8GMCSF protein.

For example, the structure of the single-stranded protein can be that, the C-terminus of hIL12b is fused to the N-terminus of hIL12a, the C-terminus of hIL12a is fused to the N-terminus of hIL2, the C-terminus of hIL2 is fused to the N-terminus of hF8V_{H}, the C-terminus of hF8V_{H} is fused to the N-terminus of hF8V_{L}, the C-terminus of hF8V_{L} is fused to the N-terminus of hF8V_{H}, the C-terminus of hF8V_{H} is fused to the N-terminus of hF8V_{L}, the C-terminus of hF8V_{L} is fused to the N-terminus of hGMCSF, thus forming an hIL12b-hIL12a-hIL2-hF8V_{H}-hF8V_{L}-hF8V_{H}-hF8V_{L}-hGMCSF protein molecule (its amino acid sequence can be as set forth in SEQ ID NO. 47), which can be referred to as hIL12bIL12aIL2DiaF8GMCSF protein.

For example, the structure of the single-stranded protein can be that, the C-terminus of hIL12b is fused to the N-terminus of hIL12a, the C-terminus of hIL12a is fused to the N-terminus of hL19V_{H}, the C-terminus of hL19V_{H} is fused to the N-terminus of hL19V_{L}, the C-terminus of hL19V_{L} is fused to the N-terminus of hL19V_{H}, the C-terminus of hL19V_{H} is fused to the N-terminus of hL19V_{L}, the C-terminus of hL19V_{L} is fused to the N-terminus of hGMCSF, and the C-terminus of hGMCSF is fused to the N-terminus of hIL2, thus forming an hIL12b-hIL12a-hL19V_{H}-hL19V_{L}-hL19V_{H}-hL19V_{L}-hGMCSF-hIL2 protein molecule (its amino acid sequence can be as set forth in SEQ ID NO. 48), which can be referred to as hIL12bIL12aDiaL19GMCSFIL2 protein.

For example, the structure of the single-stranded protein can be that, the C-terminus of hIL12b is fused to the N-terminus of hIL12a, the C-terminus of hIL12a is fused to the N-terminus of hNHS76V_{H}, the C-terminus of hNHS76V_{H} is fused to the N-terminus of hNHS76V_{L}, the C-terminus of hNHS76V_{L} is fused to the N-terminus of hNHS76V_{H}, the C-terminus of hNHS76V_{H} is fused to the N-terminus of hNHS76V_{L}, the C-terminus of hNHS76V_{L} is fused to the N-terminus of hGMCSF, and the C-terminus of hGMCSF is fused to the N-terminus of hIL2, thus forming an hIL12b-hIL12a-hNHS76V_{H}-hNHS76V_{L}-hNHS76V_{H}-hNHS76V_{L}-hGMCSF-hIL2 protein molecule (its amino acid sequence can be as set forth in SEQ ID NO. 49), which can be referred to as hIL12bIL12aDiaNHS76GMCSFIL2 protein.

For example, the structure of the single-stranded protein can be that, the C-terminus of hIL12b is fused to the N-terminus of hIL12a, the C-terminus of hIL12a is fused to the N-terminus of hNHS76V_{H}, the C-terminus of hNHS76V_{H} is fused to the N-terminus of hF8V_{L}, the C-terminus of hF8V_{L} is fused to the N-terminus of hF8V_{H}, the C-terminus of hF8V_{H} is fused to the N-terminus of hNHS76V_{L}, the C-terminus of hNHS76V_{L} is fused to the N-terminus of hGMCSF, and the C-terminus of hGMCSF is fused to the N-terminus of hIL2, thus forming an hIL12b-hIL12a-hNHS76V_{H}-hF8V_{L}-hF8V_{H}-hNHS76V_{L}-hGMCSF-hIL2 protein molecule (its amino acid sequence can be as set forth in SEQ ID NO. 50), which can be referred to as hIL12bIL12aDiaNHS76F8GMCSFIL2 protein.

In addition, in some embodiments, the single-stranded protein may also be mIL12bIL12aIL2DiaNHS76F8GMCSF-Thr (its amino acid sequence can be as set forth in SEQ ID NO. 51), which is essentially the same as the structure of the mIL12bIL12aIL2DiaNHS76F8GMCSF protein, with the difference only in the linker. The linker of mIL12bIL12aIL2DiaNHS76F8GMCSF-Thr contains a thrombin cleavage site.

In addition, in some embodiments, the single-stranded protein may also be hIL12bIL12aIL2DiaNHS76F8GMCSF-Thr (its amino acid sequence can be as set forth in SEQ ID NO. 52), which is essentially the same as the structure of the hIL12bIL12aIL2DiaNHS76F8GMCSF protein, with the difference only in the linker. The linker of hIL12bIL12aIL2DiaNHS76F8GMCSF-Thr contains a thrombin cleavage site.

In the present application, the proteins may also be dimers composed of a first polypeptide chain and a second polypeptide chain, and the first polypeptide chain is different from the second polypeptide chain.

In the present application, the first polypeptide chain can include IL12a, and the second polypeptide chain can include IL12b.

In the present application, the IL2 or a functional fragment thereof may be located in the first polypeptide chain or the second polypeptide chain, the GMCSF or a functional fragment thereof may be located in the first polypeptide chain or the second polypeptide chain.

In the present application, in the first polypeptide chain, the IL12a or the functional fragment thereof and the IL2 or the functional fragment thereof are sequentially included from N-terminus to C-terminus; alternatively, in the first polypeptide chain, the IL2 or the functional fragment thereof and the IL12a or the functional fragment thereof are sequentially included from N-terminus to C-terminus; also alternatively, in the first polypeptide chain, the IL12a or the functional fragment thereof and the GMCSF or the functional fragment thereof are sequentially included from N-terminus to C-terminus.

In the present application, in the second polypeptide chain, the IL12b or the functional fragment thereof and the GMCSF or the functional fragment thereof are sequentially included from N-terminus to C-terminus; alternatively, in the second polypeptide chain, the GMCSF or the functional fragment thereof and the IL12b or the functional fragment thereof are sequentially included from N-terminus to C-terminus; also alternatively, in the second polypeptide chain, the IL12b or the functional fragment thereof and the IL2 or the functional fragment thereof are sequentially included from N-terminus to C-terminus.

In some embodiments, in the pharmaceutical composition, the first polypeptide chain can include an amino acid sequence as set forth in SEQ ID NO. 53 and the second polypeptide chain can include an amino acid sequence as set forth in SEQ ID NO. 57; alternatively, the first polypeptide chain can include an amino acid sequence as set forth in SEQ ID NO. 54 and the second polypeptide chain can include an amino acid sequence as set forth in SEQ ID NO. 57; alternatively, the first polypeptide chain can include an amino acid sequence as set forth in SEQ ID NO. 53 and the second polypeptide chain can include an amino acid sequence as set forth in SEQ ID NO. 58; alternatively, the first polypeptide chain can include an amino acid sequence as set forth in SEQ ID NO. 54 and the second polypeptide chain can include an amino acid sequence as set forth in SEQ ID NO. 58; alternatively, the first polypeptide chain can include an amino acid sequence as set forth in SEQ ID NO. 55 and the second polypeptide chain can include an amino acid sequence as set forth in SEQ ID NO. 59; alternatively, the first polypeptide chain can include an amino acid sequence as set forth in SEQ ID NO. 56 and the second polypeptide chain can include an amino acid sequence as set forth in SEQ ID NO. 60.

In the present application, the proteins may be dimers composed of a first polypeptide chain and a second polypeptide chain.

For example, in the dimer, the C-terminus of mIL12a and the N-terminus of mIL2 can be fused to form the first polypeptide chain of mIL12a-mIL2 (its sequence is as set forth in SEQ ID NO. 53), and the C-terminus of mIL12b and the N-terminus of mGMCSF can be fused to form the second polypeptide chain of mIL12b-mGMCSF (its sequence is as set forth in SEQ ID NO. 57), thus forming an mIL12a-mIL2-mIL12b-mGMCSF protein heterodimer, which can be referred to as mIL12aIL2-IL12bGMCSF heterodimer.

For example, in the dimer, the C-terminus of mIL2 and the N-terminus of mIL12a can be fused to form the first polypeptide chain of mIL2-mIL12a (its sequence is as set forth in SEQ ID NO. 54), and the C-terminus of mIL12b and the N-terminus of mGMCSF can be fused to form the second polypeptide chain of mIL12b-mGMCSF (its sequence is as set forth in SEQ ID NO. 57), thus forming an mIL2-mIL12a-mIL12b-mGMCSF protein heterodimer, which can be referred to as mIL2IL12a-IL12bGMCSF heterodimer.

For example, in the dimer, the C-terminus of mIL12a and the N-terminus of mIL2 can be fused to form the first polypeptide chain of mIL12a-mIL2 (its sequence is as set forth in SEQ ID NO. 53), and the C-terminus of mGMCSF and the N-terminus of mIL12b can be fused to form the second polypeptide chain of mGMCSF-mIL12b (its sequence is as set forth in SEQ ID NO. 58), thus forming an mIL12a-mIL2-mGMCSF-mIL12b protein heterodimer, which can be referred to as mIL12aIL2-GMCSFIL12b heterodimer.

For example, in the dimer, the C-terminus of mIL2 and the N-terminus of mIL12a can be fused to form the first polypeptide chain of mIL2-mIL12a (its sequence is as set forth in SEQ ID NO. 54), and the C-terminus of mGMCSF and the N-terminus of mIL12b can be fused to form the second polypeptide chain of mGMCSF-mIL12b (its sequence is as set forth in SEQ ID NO. 58), thus forming an mIL2-mIL12a-mGMCSF-mIL12b protein heterodimer, which can be referred to as mIL2IL12a-GMCSFIL12b heterodimer.

For example, in the dimer, the C-terminus of mIL12a and the N-terminus of mGMCSF can be fused to form the first polypeptide chain of mIL12a-mGMCSF (its sequence is as set forth in SEQ ID NO. 55), and the C-terminus of mIL12b and the N-terminus of mIL2 can be fused to form the second polypeptide chain of mIL12b-mIL2 (its sequence is as set forth in SEQ ID NO. 59), thus forming an mIL12a-mGMCSF-mIL12b-mIL2 protein heterodimer, which can be referred to as mIL12aGMCSF-IL12bIL2 heterodimer.

For example, in the dimer, the C-terminus of hIL12a and the N-terminus of hIL2 can be fused to form a first polypeptide chain of hIL12a-hIL2 (its sequence is as set forth in SEQ ID NO. 56), and the C-terminus of hIL12b and the N-terminus of hGMCSF can be fused to form a second polypeptide chain of hIL12b-hGMCSF (its sequence is as set forth in SEQ ID NO. 60), thus forming an hIL12a-hIL2-hIL12b-hGMCSF protein heterodimer, which can be referred to as hIL12aIL2-IL12bGMCSF heterodimer.

In the present application, the IL2 or the functional fragment thereof may be located in the first polypeptide chain or the second polypeptide chain, the GMCSF or the functional fragment thereof may be located in the first polypeptide chain or the second polypeptide chain, and one or more of the targeting moieties may be each independently located in the first polypeptide chain or the second polypeptide chain.

In the present application, in the first polypeptide chain, the targeting moiety, the IL12a or the functional fragment thereof, the IL2 or the functional fragment thereof and the GMCSF or the functional fragment thereof are sequentially included from N-terminus to C-terminus; alternatively, in the first polypeptide chain, the IL2 or the functional fragment thereof, the IL12a or the functional fragment thereof and the GMCSF or the functional fragment thereof are sequentially included from N-terminus to C-terminus.

In the present application, in the second polypeptide chain, the IL12b or the functional fragment thereof and the targeting moiety are sequentially included from N-terminus to C-terminus.

In some embodiments, in the pharmaceutical composition, the first polypeptide chain can include an amino acid sequence as set forth in SEQ ID NO. 66 and the second polypeptide chain can include an amino acid sequence as set forth in SEQ ID NO. 61; alternatively, the first polypeptide chain can include an amino acid sequence as set forth in SEQ ID NO. 66 and the second polypeptide chain can include an amino acid sequence as set forth in SEQ ID NO. 62; alternatively, the first polypeptide chain can include an amino acid sequence as set forth in SEQ ID NO. 66 and the second polypeptide chain can include an amino acid sequence as set forth in SEQ ID NO. 63; alternatively, the first polypeptide chain can include an amino acid sequence as set forth in SEQ ID NO. 67 and the second polypeptide chain can include an amino acid sequence as set forth in SEQ ID NO. 61; alternatively, the first polypeptide chain can include an amino acid sequence as set forth in SEQ ID NO. 68 and the second polypeptide chain can include an amino acid sequence as set forth in SEQ ID NO. 62; alternatively, the first polypeptide chain can include an amino acid sequence as set forth in SEQ ID NO. 69 and the second polypeptide chain can include an amino acid sequence as set forth in SEQ ID NO. 63; alternatively, the first polypeptide chain can include an amino acid sequence as set forth in SEQ ID NO. 70 and the second polypeptide chain can include an amino acid sequence as set forth in SEQ ID NO. 64; alternatively, the first polypeptide chain can include an amino acid sequence as set forth in SEQ ID NO. 71 and the second polypeptide chain can include an amino acid sequence as set forth in SEQ ID NO. 64; alternatively, the first polypeptide chain can include an amino acid sequence as set forth in SEQ ID NO. 72 and the second polypeptide chain can include an amino acid sequence as set forth in SEQ ID NO. 65.

For example, in the dimer, the C-terminus of mIL12b can be fused to the N-terminus of L19V_{H} and the C-terminus of L19V_{H} can be fused to the N-terminus of L19V_{L} to form the second polypeptide chain (its amino acid sequence can be as set forth in SEQ ID NO. 61), and the C-terminus of mIL2 can be fused to the N-terminus of mIL12a and the C-terminus of mIL12a can be fused to the N-terminus of mGMCSF to form the first polypeptide chain (its amino acid sequence can be as set forth in SEQ ID NO. 66), thus forming an mIL12b-L19V_{H}-L19V_{L}-mIL2-mIL12a-mGMCSF protein heterodimer, which can be referred to as mIL12bscL19-IL2IL12aGMCSF heterodimer.

For example, in the dimer, the C-terminus of mIL12b can be fused to the N-terminus of F8V_{H} and the C-terminus of F8V_{H} can be fused to the N-terminus of F8V_{L} to form the second polypeptide chain (its amino acid sequence can be as set forth in SEQ ID NO. 62), and the C-terminus of mIL2 can be fused to the N-terminus of mIL12a and the C-terminus of mIL12a can be fused to the N-terminus of mGMCSF to form the first polypeptide chain (its amino acid sequence can be as set forth in SEQ ID NO. 66), thus forming an mIL12b-F8V_{H}-F8V_{L}-mIL2-mIL12a-mGMCSF protein heterodimer, which can be referred to as mIL12bscF8-IL2IL12aGMCSF heterodimer.

For example, in the dimer, the C-terminus of mIL12b can be fused to the N-terminus of NHS76V_{H} and the C-terminus of NHS76V_{H} can be fused to the N-terminus of NHS76V_{L} to form the second polypeptide chain (its amino acid sequence can be as set forth in SEQ ID NO. 63), and the C-terminus of mIL2 can be fused to the N-terminus of mIL12a and the C-terminus of mIL12a can be fused to the N-terminus of mGMCSF to form the first polypeptide chain (its amino acid sequence can be as set forth in SEQ ID NO. 66), thus forming an mIL12b-NHS76V_{H}-NHS76V_{L}-mIL2-mIL12a-mGMCSF protein heterodimer, which can be referred to as mIL12bscNHS76-IL2IL12aGMCSF heterodimer.

For example, in the dimer, the C-terminus of mIL12b can be fused to the N-terminus of L19V_{H} and the C-terminus of L19V_{H} can be fused to the N-terminus of L19V_{L} to form the second polypeptide chain (its amino acid sequence can be as set forth in SEQ ID NO. 61), and the C-terminus of L19V_{H} can be fused to the N-terminus of L19V_{L}, the C-terminus of L19V_{L} can be fused to the N-terminus of mIL12a, the C-terminus of mIL12a can be fused to the N-terminus of mIL2, and the C-terminus of mIL2 can be fused to the N-terminus of mGMCSF to form the first polypeptide chain (its amino acid sequence can be as set forth in SEQ ID NO. 67), thus forming an mIL12b-L19V_{H}-L19V_{L}-L19V_{H}-L19V_{L}-mIL12a-mIL2-mGMCSF protein heterodimer, which can be referred to as mIL12bscL19-scL19IL12aIL2GMCSF heterodimer.

For example, in the dimer, the C-terminus of mIL12b can be fused to the N-terminus of F8V_{H} and the C-terminus of F8V_{H} can be fused to the N-terminus of F8V_{L} to form the second polypeptide chain (its amino acid sequence can be as set forth in SEQ ID NO. 62), and the C-terminus of F8V_{H} can be fused to the N-terminus of F8V_{L}, the C-terminus of F8V_{L} can be fused to the N-terminus of mIL12a, the C-terminus of mIL12a can be fused to the N-terminus of mIL2, and the C-terminus of mIL2 can be fused to the N-terminus of mGMCSF to form the first polypeptide chain (its amino acid sequence can be as set forth in SEQ ID NO. 68), thus forming an mIL12b-F8V_{H}-F8V_{L}-F8V_{H}-F8V_{L}-mIL12a-mIL2-mGMCSF protein heterodimer, which can be referred to as mIL12bscF8-scF8IL12aIL2GMCSF heterodimer.

For example, in the dimer, the C-terminus of mIL12b can be fused to the N-terminus of NHS76V_{H} and the C-terminus of NHS76V_{H} can be fused to the N-terminus of NHS76V_{L} to form the second polypeptide chain (its amino acid sequence can be as set forth in SEQ ID NO. 63), and the C-terminus of NHS76V_{H} can be fused to the N-terminus of NHS76V_{L}, the C-terminus of NHS76V_{L} can be fused to the N-terminus of mIL12a, the C-terminus of mIL12a can be fused to the N-terminus of mIL2, and the C-terminus of mIL2 can be fused to the N-terminus of GMCSF to form the first polypeptide chain (its amino acid sequence can be as set forth in SEQ ID NO. 69), thus forming an mIL12b-NHS76V_{H}-NHS76V_{L}-NHS76V_{H}-NHS76V_{L}-mIL12a-mIL2-mGMCSF protein heterodimer, which can be referred to as mIL12bscNHS76-scNHS76IL12aIL2GMCSF heterodimer.

For example, in the dimer, the C-terminus of hIL12b can be fused to the N-terminus of F8V_{H} and the C-terminus of F8V_{H} can be fused to the N-terminus of F8V_{L} to form the second polypeptide chain (its amino acid sequence can be as set forth in SEQ ID NO. 64), and the C-terminus of hIL2 can be fused to the N-terminus of hIL12a and the C-terminus of hIL12a can be fused to the N-terminus of hGMCSF to form the first polypeptide chain (its amino acid sequence can be as set forth in SEQ ID NO. 70), thus forming an hIL12b-F8V_{H}-F8V_{L}-hIL2-hIL12a-hGMCSF protein heterodimer, which can be referred to as hIL12bscF8-IL2IL12aGMCSF heterodimer.

For example, in the dimer, the C-terminus of hIL12b can be fused to the N-terminus of F8V_{H} and the C-terminus of F8V_{H} can be fused to the N-terminus of F8V_{L} to form the second polypeptide chain (its amino acid sequence can be as set forth in SEQ ID NO. 64), and the C-terminus of F8V_{H} can be fused to the N-terminus of F8V_{L}, the C-terminus of F8V_{L} can be fused to the N-terminus of hIL12a, the C-terminus of hIL12a can be fused to the N-terminus of hIL2, and the C-terminus of hIL2 can be fused to the N-terminus of hGMCSF to form the first polypeptide chain (its amino acid sequence can be as set forth in SEQ ID NO. 71), thus forming an hIL12b-F8V_{H}-F8V_{L}-F8V_{H}-F8V_{L}-hIL12a-hIL2-hGMCSF protein heterodimer, which can be referred to as hIL12bscF8-scF8IL12aIL2GMCSF heterodimer.

For example, in the dimer, the C-terminus of hIL12b can be fused to the N-terminus of NHS76V_{H} and the C-terminus of NHS76V_{H} can be fused to the N-terminus of NHS76V_{L} to form the second polypeptide chain (its amino acid sequence can be as set forth in SEQ ID NO. 65), and the C-terminus of NHS76V_{H} can be fused to the N-terminus of NHS76V_{L}, the C-terminus of NHS76V_{L} can be fused to the N-terminus of hIL12a, the C-terminus of hIL12a can be fused to the N-terminus of hIL2, and the C-terminus of hIL2 can be fused to the N-terminus of hGMCSF to form the first polypeptide chain (its amino acid sequence can be as set forth in SEQ ID NO. 72), thus forming an hIL12b-NHS76V_{H}-NHS76V_{L}-NHS76V_{H}-NHS76V_{L}-hIL12a-hIL2-hGMCSF protein heterodimer, which can be referred to as hIL12bscNHS76-scNHS76IL12aIL2GMCSF heterodimer.

In another aspect, the present application provides a reagent kit including the pharmaceutical composition of the present application. The reagent kit can include a box or container containing the components of the reagent kit. The box or container is attached with a label or a FDA-approved treatment regimen. The box or container contains the components of the pharmaceutical composition of the present application, for example, the components can be contained in a plastic, polyethylene, polypropylene, ethylene or propylene container. The container can be a capped tube or bottle. In addition, the reagent kit can also contain instructions for administering the pharmaceutical composition of the present application.

Uses

In another aspect, the present application further provides uses of the pharmaceutical composition or the reagent kit in preparing a medicament for treating a tumor.

In the present application, the pharmaceutical composition or the reagent kit can be used for treating a tumor.

In another aspect, the present application further provides a method for treating a tumor, which includes administering to a subject in need thereof the pharmaceutical composition or the reagent kit.

In the present application, the tumor may include a solid tumor or a non-solid tumor. For example, the tumor may include melanoma. For example, the tumor may include breast cancer. For example, the tumor may include lung cancer.

In the present application, the administration may include administering the proteins first, and then administering the immune checkpoint inhibitor. For example, the administration may include administering the single-stranded protein of the present application first, and then administering the immune checkpoint inhibitor of the present application. Further for example, the administration may include administering the dimer of the present application first, and then administering the immune checkpoint inhibitor of the present application.

In the present application, the administration may include intratumoral injection. For example, the pharmaceutical composition of the present application is injected intratumorally. In some embodiments, the administration method can also be oral administration, intravenous administration, intramuscular administration, in situ administration at the tumor site, inhalation, rectal administration, vaginal administration, transdermal administration, or administration via a subcutaneous reservoir. In some embodiments, the administration can also include intravenous injection or subcutaneous injection.

In some embodiments, the pharmaceutical composition of the present application can be formulated for oral administration, intravenous administration, intramuscular administration, in situ administration at the tumor site, inhalation, rectal administration, vaginal administration, transdermal administration, or administration via a subcutaneous reservoir.

In some embodiments, the pharmaceutical composition of the present application can also include a pharmaceutically acceptable carrier. For example, the pharmaceutically acceptable carrier may include buffers, antioxidants, preservatives, low molecular weight polypeptides, proteins, hydrophilic polymers, amino acids, sugars, chelating agents, counterions, metal complexes and/or nonionic surfactants, etc. For example, the pharmaceutically acceptable carrier may include excipients. For example, the excipients may be selected from the following group: starch, dextrin, sucrose, lactose, magnesium stearate, calcium sulfate, carboxymethyl cellulose, talc, calcium alginate gel, chitosan, and nanospheres, etc. For example, the pharmaceutically acceptable carrier may also be selected from the following group: a pH regulator, an osmotic pressure regulator, a solubilizer and a bacteriostatic agent.

Without intending to be limited by any theory, the following examples are only intended to illustrate the pharmaceutical composition and uses thereof, and are not intended to limit the scope of the present invention.

### EXAMPLES

Reagents: DMEM medium, 1640 medium and fetal calf serum, purchased from Life Technologies Corporation; Cell culture flasks and culture plates, purchased from Corning Incorporated; Doxycycline, purchased from Sangon Biotech (Shanghai) Co., Ltd.; Puromycin and Blasticidin, purchased from Chemicon Corporation; Restriction enzyme, purchased from Takara and NEB, Inc.; Ligase, purchased from NEB, Inc.; DNA polymerase, purchased from Takara Bio Inc.; Plasmid extraction kit and Gel extraction kit, purchased from OmegaBiotech, Inc.; primer synthesis, completed by Sangon Biotech (Shanghai) Co., Ltd.; gene synthesis and sequencing, completed by Life Technologies Corporation; Antibodies for flow cytometry, purchased from Ebioscience, Inc.; PD1 blocking antibodies, purchased from BioXcell Inc.; Protein Magnetic Bead Purification Kit, purchased from BeaverBio.

### Example 1. Expression of PD1 in T cells induced by mIL12aIL2-IL12bGMCSF heterodimer

1.1 Construction of the First Expression Vector

The DNA sequence of rtTA was synthesized, which has BamHI and EcoRI sites respectively at its two ends, and the synthesized product was ligated within a vector pUC57. The vector was digested by an enzyme digestion system as below: plasmid 6 μg, digestion buffer 4 μl, BamHI 1 μl, EcoRI 1 μl, adding water to a total volume of 40 μl, and standing at 37° C. for 12 hours. The EP tube was taken out, into which was added 4.4 μl of 10× loading buffer. Electrophoresis was conducted with 1% sepharose gel, after then rtTA fragments were recycled, and ready for use.

The vector pLentis-CMV-MES-Bsd was digested in an EP tube by an enzyme digestion system as below: plasmid 2 μg, digestion buffer 3 μl, BamHI 1 μl, EcoRI 1 μl, adding water to a total volume of 30 μl, and standing at 37° C. for 12 hours. The EP tube was taken out, into which was added 3.3 μl of 10× loading buffer. Electrophoresis was conducted with 1% sepharose gel, after then pLentis-CMV-IRES-Bsd vector fragments were recycled, and ready for use.

pLentis-CMV-IRES-Bsd and rtTA were ligated by a system as below: pLentis-CMV-IRES-Bsd 2 μl, rtTA 2 μl, ligase buffer 1 μl, T4DNA ligase 0.5 μl, water 4.5 μl, at room temperature for 4 hours. The ligation system was then subjected to the transformation of Escherichia coli competence. The next day, colonies were picked from the transformed plates, and cultured in LB medium at 37° C. in a shaking bed overnight. Plasmids were extracted from the cultured bacteria using a plasmid extraction kit, and digested to identify whether the fragments had been successfully ligated into the vector or not. The correct vectors were then sent for sequencing to determine the successful construction of the first expression vector pLentis-CMV-rtTA-IRES-Bsd.

1.2 Acquisition of Cells containing the First Expression Vector

First, the viruses of the first expression vector were prepared as below:

1. The digested and cultured 293FT cells were counted and then spread into a 10 cm culture dish at 3×10⁶ cells/well in which the volume of the culture solution was 10 ml.

2. On the evening of the next day, the state of the cells was observed, and the cells were transfected if they were in good and suitable state. Chloroquine was added into the culture plate to a final concentration of 25 μM. One test tube was taken, into which were added sterile water and the following plasmids (pMD2.G 5 μg+pSPAX2 15 μg+pLentis-CMV-rtTA-IRES-Bsd 20 μg) to a total volume of 1045 μl, then added 155 μl of 2M CaCl₂ and mixed well, and finally added 1200 μl of 2×HBS dropwise while shaking. After the completion of the dropwise addition, the mixture was immediately added into the cell culture wells and mixed well by gently shaking.

3. On the morning of the third day, the state of the cells was observed, and the medium was replaced to 10 ml of fresh DMEM medium.

4. On the morning of the fifth day, the state of the cells was observed, and the supernatant in the culture dish was collected, filtered through a 0.45 μm filter, and centrifuged in a high-speed centrifuge tube at 50000 g for 2 hours. The supernatant was discarded carefully. The liquid was drained as much as possible with absorbent paper. The residues were then resuspended with 500 μl of HBSS and dissolved for 2 hours, then dispensed into small tubes and stored at −70° C.

Subsequently, the tumor cells were transfected with the viruses of the first expression vector as below:

The digested and cultured mouse melanoma cells B16 were seeded into a 6-well plate at 10⁵ cells/well in a culture volume of 1 ml. 24 hours later, 10 μl of the viruses of the first expression vector were added and the cells were further cultured in the incubator for 24 hours. After then, the supernatant was discarded, and the culture was continued after replacing with a fresh medium. After the cells were confluent, they were transferred into a culture flask, into which blasticidin was added at a concentration suitable for the cells to continue the culture. The culture medium was replaced every two days, and the concentration of blasticidin was kept at 8 μg/ml. After one week of screening, the surviving cells were those that stably expressed the regulatory proteins, and the cells were named as B16 (rtTA).

1.3 Construction of the Second Expression Vector Encoding the mIL12aIL2-IL12bGMCSF Heterodimer

The gene of the mIL12aIL2-IL12bGMCSF heterodimer was synthesized, which had BamHI and EcoRI cleavage sites respectively at its two ends. The synthesized gene was then digested with BamHI and EcoRI by an enzyme digestion system as below: mIL12aIL2-IL12bGMCSF heterodimer plasmid 5 μg, digestion buffer 4 μl, BamHI 1 μl, EcoRI 1 μl, adding water to a total volume of 40 μl, and standing at 37° C. for 12 hours. The EP tube was taken out, into which was added 4.4 μl of 10× loading buffer. Electrophoresis was conducted with 1% sepharose gel, after then mIL12aIL2-IL12bGMCSF gene fragments were recycled, and ready for use.

For the mIL12aIL2-IL12bGMCSF heterodimer, the amino acid sequence of the first polypeptide chain is as set forth in SEQ ID NO. 53, and the amino acid sequence of the second polypeptide chain is as set forth in SEQ ID NO. 57; the nucleotide sequence encoding the mIL12aIL2-IL12bGMCSF is as set forth in SEQ ID NO. 73.

The regulatory expression vector pLentis-PTRE-MCS-PGK-PURO was digested by an enzyme digestion system as below: pLentis-PTRE-MCS-PGK-PURO plasmid 2 μg, digestion buffer 3 μl, BamHI 1 μl, EcoRI 1 μl, adding water to a total volume of 30 μl, and standing at 37° C. for 12 hours. The EP tube was taken out, into which was added 3.3 μl of 10× loading buffer. Electrophoresis was conducted with 1% sepharose gel, after then pLentis-PTRE-MCS-PGK-PURO vector fragments were recycled, and ready for use.

pLentis-PTRE-MCS-PGK-PURO and IL2 were ligated by a system as below: pLentis-PTRE-MCS-PGK-PURO 2 μl, mIL12aIL2-IL12bGMCSF 2 μl, ligase buffer 1 μl, T4DNA ligase 0.5 μl, water 4.5 μl, at room temperature for 4 hours. The ligation system was then subjected to the transformation of Escherichia coli competence. The next day, colonies were picked from the transformed plates, and cultured in LB medium at 37° C. in a shaking bed overnight. Plasmids were extracted from the cultured bacteria using a plasmid extraction kit, and digested to identify whether the fragments had been successfully ligated into the vector or not. The correct vectors were then sent for sequencing to determine the successful construction of the second expression vector pLentis-PTRE-mIL12aIL2-IL12bGMCSF-PGK-PURO.

1.4 Preparation of Cells that Regulate the Expression of mIL12aIL2-IL12bGMCSF Heterodimer

The viruses of the expression vector of mIL12aIL2-IL12bGMCSF heterodimer were prepared by a method that was consistent with the preparation method of the viruses of the first expression vector. The digested and cultured B16 (rtTA) tumor cells were seeded into a 6-well plate at 10⁵ cells/well in a culture volume of 1 ml. 24 hours later, 10 μl of the viruses of the regulatory expression vector (i.e., the viruses of the expression vector of mIL12aIL2-IL12bGMCSF heterodimer) were added and the cells were further cultured in the incubator for 24 hours. After then, the supernatant was discarded, and the culture was continued after replacing with a fresh medium. After the cells were confluent, they were transferred into a culture flask, into which puromycin was added to a final concentration of 3 μg/ml. After continuing the culture for three days, the surviving cells were those that can regulate the expression of mIL12aIL2-IL12bGMCSF, and the cells were named as B16(rtTA)-mIL12aIL2-IL12bGMCSF.

1.5 Analysis on the Expression of PD1 in T Cells after Inducing the Expression of mIL12aIL2-IL12bGMCSF Heterodimer

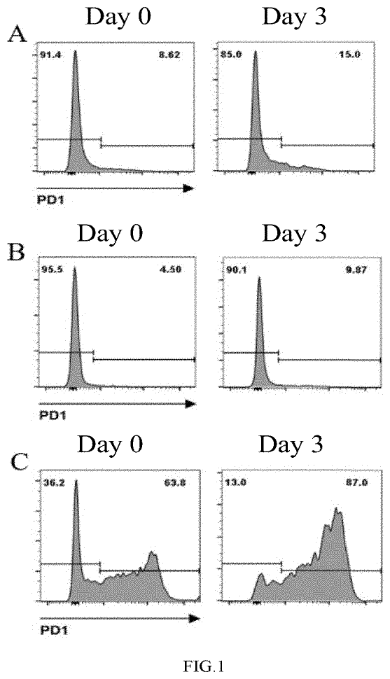

B16(rtTA)-mIL12aIL2-IL12bGMCSF cells in the logarithmic growth phase were digested and diluted with HBSS to 2×10⁶/ml. The cells were injected using a 1 ml syringe into the right dorsal side of 8-10-week-old C57BL/6 female mice at 50 μl per mouse, with a total of 10 mice. After tumor growth, the mice were fed with water containing 2 g/L of doxycycline. Mice on day 0 and day 3 after the addition of doxycycline were taken, from which spleen cells and tumor tissue cells were isolated respectively. After lysing red blood cells with red blood cell lysis buffer, the cells were filtered through a sieve to obtain a single cell suspension. The single cell suspension was stained with CD45 antibodies, CD3 antibodies, CD4 antibodies, CD8 antibodies, and PD1 antibodies, and then washed with PBS twice. A flow cytometer was used for analysis to determine the changes of the expression of PD1 in T cells. The results were shown in FIG. 1, wherein FIG. 1A, FIG. 1B and FIG. 1C showed the expression of PD1 in spleenic CD4 T cells, spleenic CD8 T cells and intratumoral CD3 T cells of mice, respectively. It can be seen that, the expression of PD1 in spleenic CD4 T cells, CD8 T cells and tumor infiltrating CD3 T cells of mice was all significantly elevated.

### Example 2. Expression of mIL12bIL12aIL2GMCSF Single-Stranded Protein

2.1 Construction of Expression Vector

6*His were added at the termini of the single-stranded protein molecule mIL12bIL12aIL2GMCSF for ease of purification. The corresponding DNA sequence of the gene was synthesized. The synthesized sequence had BamHI and XhoI cleavage sites at the front and back ends, respectively. The synthetic plasmids containing the target gene were digested by a system as below: 5 μg of plasmid, 4 μl of digestion buffer, 1 μl of BamHI and 1 μl of XhoI, adding water to a total volume of 40 μl, and standing at 37° C. for 12 hours. The EP tube was taken out, into which was added 4.4 μl of 10× loading buffer. Electrophoresis was conducted with 1% sepharose gel, after then mIL12bIL12aIL2GMCSF protein gene fragments were recycled, and ready for use.

The amino acid sequence of the mIL12bIL12aIL2GMCSF single-stranded protein is as set forth in SEQ ID NO. 27, and the nucleotide sequence encoding the mIL12bIL12aIL2GMCSF is as set forth in SEQ ID NO. 74.

The vector pLentis-CMV-MCS-IRES-PURO was digested in an EP tube by a system as below: 2 μg of pLentis-CMV-MCS-IRES-PURO vector plasmid, 3 μl of digestion buffer, 1 μl of BamHI and 1 μl of XhoI, adding water to a total volume of 30 μl, and standing at 37° C. for 12 hours. The EP tube was taken out, into which was added 3.3 μl of 10× loading buffer. Electrophoresis was conducted with 1% sepharose gel, after then pLentis-CMV-MCS-IRES-PURO vector fragments were recycled, and ready for use.

mIL12bIL12aIL2GMCSF and pLentis-CMV-MCS-IRES-PURO were ligated by a system as below: 2 μl of pLentis-CMV-MCS-IRES-PURO vector fragment, 2 μl of mIL12bIL12aIL2GMCSF gene fragment, 1 μl of ligase buffer, 0.5 μl of T4 DNA ligase, and 4.5 μl of water, at room temperature for 4 hours. The ligation system was then subjected to the transformation of Escherichia coli competence. The next day, colonies were picked from the transformed plate, and cultured in LB medium at 37° C. in a shaking bed overnight. Plasmids were extracted from the cultured bacteria using a plasmid extraction kit, and digested to identify whether the fragments had been successfully ligated into the vector or not. The correct vectors were then sent for sequencing to determine the successful construction. The expression vector pLentis-CMV-mIL12bIL12aIL2GMCSF-IRES-PURO was obtained.

2.2 Preparation of Expression Viruses

1) The digested and cultured 293FT cells were counted and then spread into a 10 cm culture dish at 3×10⁶ cells/well in which the volume of the culture solution was 10 ml.

2) On the evening of the next day, the state of the cells was observed, and the cells were transfected if they were in good condition. Chloroquine was added into the culture plate to a final concentration of 25 μM. One test tube was taken, into which were added sterile water and the following plasmids (6 μg of pMD2.G+15 μg of pSPAX2+20 μg of the expression vector obtained in the above example 2.1) to a total volume of 1045 μl, then added 155 μl of 2M CaCl₂ and mixed well, and finally added 1200 μl of 2×HBS dropwise while shaking. After the completion of the dropwise addition, the mixture was immediately added into the cell culture wells and mixed well by gently shaking.

3) On the morning of the third day, the state of the cells was observed, and the medium was replaced to 10 ml of fresh DMEM medium.

4) On the morning of the fifth day, the state of the cells was observed, and the supernatant in the culture dish was collected, filtered through a 0.45 μm filter, and centrifuged in a high-speed centrifuge tube at 50000 g for 2 hours. The supernatant was discarded carefully. The liquid was drained as much as possible with absorbent paper. The residues were then resuspended with 200 μl of HBSS and dissolved for 2 hours, then dispensed into small tubes and stored at −70° C.

2.3 Preparation of Expression Cells

The digested and cultured 293A cells were seeded into a 6-well plate at 10⁵ cells/well in a culture volume of 1 ml. 24 hours later, 10 μl of the viruses expressing the above target gene (i.e., the viruses obtained in Example 2.2) were added and the cells were further cultured in the incubator for 24 hours. After then, the supernatant was discarded, and the culture was continued after replacing with a fresh medium. After the cells were confluent, they were transferred into a culture flask, into which puromycin was added at a final concentration of 3 μg/ml to continue the culture. The culture medium was replaced every two days, and the concentration of puromycin was kept. After one week of screening, the surviving cells were those that stably expressed the proteins, and the cells were named as 293A-mIL12bIL12aIL2GMCSF.

2.4 Expression and Purification of Proteins

The constructed cells 293A-mIL12bIL12aIL2GMCSF expressing mIL12bIL12aIL2GMCSF were passaged into a 15 cm culture dish. After the cells were confluent, the culture medium was replaced to 30 ml of CDM4HEK293 to continue the culture for 5 days. The supernatant was then collected, filtered through a 0.45 μm filter, and concentrated by ultrafiltration with 50 kd of AMICON ULTRA-15. The concentrated protein solution obtained was purified with nickel-chelated magnetic beads (purchased from Beaver Biosciences Inc.) according to the instructions. The purified protein solution obtained was then ultrafiltered through an AMICON ULTRA-0.5 ultrafiltration tube, with the buffer being replaced to PBS. The finally obtained protein solution was tested with an IL12p70 ELISA kit for the protein concentration. After adjusting the protein concentration to 2 μg/μl with PBS, the protein solution was dispensed and stored at −20° C.

### Example 3. Expression of mIL12bIL12aIL2DiaNHS76F8GMCSF Single-Stranded Protein

3.1 Construction of Expression Vector

6*His were added at the termini of the single-stranded protein molecule mIL12bIL12aIL2DiaNHS76F8GMCSF for ease of purification. The corresponding DNA sequence of the gene was synthesized. The synthesized sequence had BamHI and XhoI cleavage sites at the front and back ends, respectively. The synthetic plasmids containing the target gene were digested by a system as below: 5 μg of plasmid, 4 μl of digestion buffer, 1 μl of BamHI and 1 μl of XhoI, adding water to a total volume of 40 μl, and standing at 37° C. for 12 hours. The EP tube was taken out, into which was added 4.4 μl of 10× loading buffer. Electrophoresis was conducted with 1% sepharose gel, after then mIL12bIL12aIL2DiaNHS76F8GMCSF protein gene fragments were recycled, and ready for use.

The amino acid sequence of the mIL12bIL12aIL2DiaNHS76F8GMCSF single-stranded protein is as set forth in SEQ ID NO. 39, and the nucleotide sequence encoding the mIL12bIL12aIL2DiaNHS76F8GMCSF is as set forth in SEQ ID NO. 75.

The vector pLentis-CMV-MCS-IRES-PURO was digested in an EP tube by a system as below: 2 μg of pLentis-CMV-MCS-IRES-PURO vector plasmid, 3 μl of digestion buffer, 1 μl of BamHI and 1 μl of XhoI, adding water to a total volume of 30 μl, and standing at 37° C. for 12 hours. The EP tube was taken out, into which was added 3.3 μl of 10× loading buffer. Electrophoresis was conducted with 1% sepharose gel, after then pLentis-CMV-MCS-IRES-PURO vector fragments were recycled, and ready for use.

mIL12bIL12aIL2DiaNHS76F8GMCSF and pLentis-CMV-MCS-IRES-PURO were ligated by a system as below: 2 μl of pLentis-CMV-MCS-IRES-PURO vector fragment, 2 μl of mIL12bIL12aIL2DiaNHS76F8GMCSF gene fragment, 1 μl of ligase buffer, 0.5 μl of T4 DNA ligase, and 4.5 μl of water, at room temperature for 4 hours. The ligation system was then subjected to the transformation of Escherichia coli competence. The next day, colonies were picked from the transformed plate, and cultured in LB medium at 37° C. in a shaking bed overnight. Plasmids were extracted from the cultured bacteria using a plasmid extraction kit, and digested to identify whether the fragments had been successfully ligated into the vector or not. The correct vectors were then sent for sequencing to determine the successful construction. The expression vector pLentis-CMV-mIL12bIL12aIL2DiaNHS76F8GMCSF-IRES-PURO was obtained.

3.2 Preparation of Expression Viruses

1) The digested and cultured 293FT cells were counted and then spread into a 10 cm culture dish at 3×10⁶ cells/well in which the volume of the culture solution was 10 ml.

2) On the evening of the next day, the state of the cells was observed, and the cells were transfected if they were in good condition. Chloroquine was added into the culture plate to a final concentration of 25 μM. One test tube was taken, into which were added sterile water and the following plasmids (6 μg of pMD2.G+15 μg of pSPAX2+20 μg of the expression vector obtained in the above example 3.1) to a total volume of 1045 μl, then added 155 μl of 2M CaCl₂ and mixed well, and finally added 1200 μl of 2×HBS dropwise while shaking. After the completion of the dropwise addition, the mixture was immediately added into the cell culture wells and mixed well by gently shaking.

3) On the morning of the third day, the state of the cells was observed, and the medium was replaced to 10 ml of fresh DMEM medium.

4) On the morning of the fifth day, the state of the cells was observed, and the supernatant in the culture dish was collected, filtered through a 0.45 μm filter, and centrifuged in a high-speed centrifuge tube at 50000 g for 2 hours. The supernatant was discarded carefully. The liquid was drained as much as possible with absorbent paper. The residues were then resuspended with 200 μl of HBSS and dissolved for 2 hours, then dispensed into small tubes and stored at −70° C.

3.3 Preparation of Expression Cells

The digested and cultured 293A cells were seeded into a 6-well plate at 10⁵ cells/well in a culture volume of 1 ml. 24 hours later, 10 μl of the viruses expressing the above target gene (i.e., the viruses obtained in Example 3.2) were added and the cells were further cultured in the incubator for 24 hours. After then, the supernatant was discarded, and the culture was continued after replacing with a fresh medium. After the cells were confluent, they were transferred into a culture flask, into which puromycin was added at a final concentration of 3 μg/ml to continue the culture. The culture medium was replaced every two days, and the concentration of puromycin was kept. After one week of screening, the surviving cells were those that stably expressed the proteins, and the cells were named as 293A-mIL12bIL12aIL2DiaNHS76F8GMCSF.

3.4 Expression and Purification of Proteins

The constructed cells 293A-mIL12bIL12aIL2DiaNHS76F8GMCSF expressing mIL12bIL12aIL2DiaNHS76F8GMCSF were passaged into a 15 cm culture dish. After the cells were confluent, the culture medium was replaced to 30 ml of CDM4HEK293 to continue the culture for 5 days. The supernatant was then collected, filtered through a 0.45 μm filter, and concentrated by ultrafiltration with 50 kd of AMICON ULTRA-15. The concentrated protein solution obtained was purified with nickel-chelated magnetic beads (purchased from Beaver Biosciences Inc.) according to the instructions. The purified protein solution obtained was then ultrafiltered through an AMICON ULTRA-0.5 ultrafiltration tube, with the buffer being replaced to PBS. The finally obtained protein solution was tested with an IL12p70 ELISA kit for the protein concentration. After adjusting the protein concentration to 2 μg/μl with PBS, the protein solution was dispensed and stored at −20° C.

### Example 4. Effect of the Combination Treatment of mIL12bIL12aIL2GMCSF and PD1 Antibody on the Growth of Melanoma in Mice

2×10⁵ digested and cultured mouse melanoma cells (B16) were injected subcutaneously into the right side of the body of C57BL/6 mice (6-10-week-old, female), and treatment was initiated when the long diameter of the tumor reached 6-8 mm.

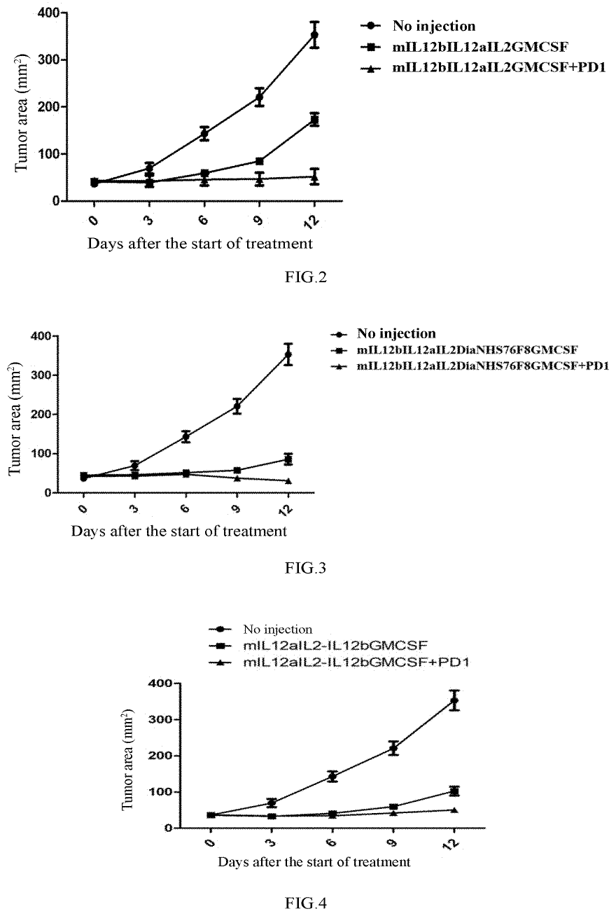

25 μl of the protein solution prepared in Example 2 was taken and added into 50 μl of glycerol, and mixed well immediately with a pipette tip to avoid air bubbles to obtain an injection preparation. The formulated injection solution was drawn with a 29G insulin syringe and slowly injected into the tumor, leaving the needle in place for a little time after the injection to reduce the overflow of the solution. The injected mice were returned to their cages, and the tumor growth of the mice was recorded. This experiment was divided into 3 groups: 1. no injection group, 2. protein solution injection group, 3. protein solution injection and PD1 antibody treatment group (i.e., combined treatment group). Wherein, in the combined treatment group, 200 μg of PD1 antibody (specifically, BioXcell Inc., InVivoMAb anti-mouse PD-1 (CD279), Item No. BE0146) was injected intraperitoneally into the mice every 3 days starting from the second day after the injection of the protein solution, for a total of 4 injections. Finally, the tumor growth in each group of mice was compared. The results were shown in FIG. 2, from which it can be seen that the combined treatment of mIL12bIL12aIL2GMCSF and PD1 antibody significantly inhibited the growth of tumor.

### Example 5. Effect of the Combination Treatment of mIL12bIL12aIL2DiaNHS76F8GMCSF and PD1 Antibody on the Growth of Melanoma in Mice

2×10⁵ digested and cultured mouse melanoma cells (B16) were injected subcutaneously into the right side of the body of C57BL/6 mice (6-10-week-old, female), and treatment was initiated when the long diameter of the tumor reached 6-8 mm.

25 μl of the protein solution prepared in Example 3 was taken and adjusted with PBS to a total volume of 200 μl. The protein solution was drawn with a 29G insulin syringe and injected into the tail vein of the mice once a day, for a total of 5 injections. The injected mice were returned to their cages, and the tumor growth of the mice was recorded. This experiment was divided into 3 groups: 1. no injection group, 2. protein solution injection group, 3. protein solution injection and PD1 antibody treatment group (i.e., combined treatment group). Wherein, in the combined treatment group, 200 μg of PD1 antibody (specifically, BioXcell Inc., InVivoMAb anti-mouse PD-1 (CD279), Item No. BE0146) was injected intraperitoneally into the mice every 3 days starting from the second day after the completion of 5 injections of the protein solution, for a total of 4 injections. Finally, the tumor growth in each group of mice was compared. The results were shown in FIG. 3, from which it can be seen that the combined treatment of mIL12bIL12aIL2DiaNHS76F8GMCSF and PD1 antibody significantly inhibited the growth of tumor.

### Example 6. Expression of mIL12aIL2-IL12bGMCSF Heterodimer Protein

6.1 Construction of Expression Vector

The gene of the mIL12aIL2-IL12bGMCSF heterodimer was synthesized. 6*His sequences were added to the C-terminus of the IL2 gene for the subsequent protein purification. The synthesized gene had BamHI and XhoI cleavage sites respectively at its two ends. It was then digested with BamHI and XhoI by an enzyme digestion system as below: mIL12aIL2-IL12bGMCSF heterodimer plasmid 5 μg, digestion buffer 4 μl, BamHI 1 μl, XhoI 1 μl, adding water to a total volume of 40 μl, and standing at 37° C. for 12 hours. The EP tube was taken out, into which was added 4.4 μl of 10× loading buffer. Electrophoresis was conducted with 1% sepharose gel, after then mIL12aIL2-IL12bGMCSF gene fragments were recycled, and ready for use.

For the mIL12aIL2-IL12bGMCSF heterodimer, the amino acid sequence of the first polypeptide chain is as set forth in SEQ ID NO. 53, and the amino acid sequence of the second polypeptide chain is as set forth in SEQ ID NO. 57; the nucleotide sequence encoding the mIL12aIL2-IL12bGMCSF is as set forth in SEQ ID NO. 73.

The vector pLentis-CMV-MCS-IRES-PURO was digested in an EP tube by a system as below: 2 μg of pLentis-CMV-MCS-IRES-PURO vector plasmid, 3 μl of digestion buffer, 1 μl of BamHI and 1 μl of XhoI, adding water to a total volume of 30 μl, and standing at 37° C. for 12 hours. The EP tube was taken out, into which was added 3.3 μl of 10× loading buffer. Electrophoresis was conducted with 1% sepharose gel, after then pLentis-CMV-MCS-IRES-PURO vector fragments were recycled, and ready for use.

mIL12aIL2-IL12bGMCSF and pLentis-CMV-MCS-IRES-PURO were ligated by a system as below: 2 μl of pLentis-CMV-MCS-IRES-PURO vector fragment, 2 μl of mIL12aIL2-IL12bGMCSF gene fragment, 1 μl of ligase buffer, 0.5 μl of T4 DNA ligase, and 4.5 μl of water, leaving at room temperature to ligate for 4 hours. The ligation system was then subjected to the transformation of Escherichia coli competence. The next day, colonies were picked from the transformed plate, and cultured in LB medium at 37° C. in a shaking bed overnight. Plasmids were extracted from the cultured bacteria using a plasmid extraction kit, and digested to identify whether the fragments had been successfully ligated into the vector or not. The correct vectors were then sent for sequencing to determine the successful construction. The expression vector pLentis-CMV-mIL12aIL2-IL12bGMCSF-IRES-PURO was obtained.

6.2 Preparation of Expression Viruses

1) The digested and cultured 293FT cells were counted and then spread into a 10 cm culture dish at 3×10⁶ cells/well in which the volume of the culture solution was 10 ml.

2) On the evening of the next day, the state of the cells was observed, and the cells were transfected if they were in good condition. Chloroquine was added into the culture plate to a final concentration of 25 μM. One test tube was taken, into which were added sterile water and the following plasmids (6 μg of pMD2.G+15 μg of pSPAX2+20 μg of the expression vector obtained in the above example 2.1) to a total volume of 1045 μl, then added 155 μl of 2M CaCl₂ and mixed well, and finally added 1200 μl of 2×HBS dropwise while shaking. After the completion of the dropwise addition, the mixture was immediately added into the cell culture wells and mixed well by gently shaking.

3) On the morning of the third day, the state of the cells was observed, and the medium was replaced to 10 ml of fresh DMEM medium.

4) On the morning of the fifth day, the state of the cells was observed, and the supernatant in the culture dish was collected, filtered through a 0.45 μm filter, and centrifuged in a high-speed centrifuge tube at 50000 g for 2 hours. The supernatant was discarded carefully. The liquid was drained as much as possible with absorbent paper. The residues were then resuspended with 200 μl of HBSS and dissolved for 2 hours, then dispensed into small tubes and stored at −70° C.

6.3 Preparation of Expression Cells

The digested and cultured 293A cells were seeded into a 6-well plate at 10⁵ cells/well in a culture volume of 1 ml. 24 hours later, 10 μl of the viruses expressing the above target gene (i.e., the viruses obtained in Example 2.2) were added and the cells were further cultured in the incubator for 24 hours. After then, the supernatant was discarded, and the culture was continued after replacing with a fresh medium. After the cells were confluent, they were transferred into a culture flask, into which puromycin was added at a final concentration of 3 μg/ml to continue the culture. The culture medium was replaced every two days, and the concentration of puromycin was kept. After one week of screening, the surviving cells were those that stably expressed the proteins, and the cells were named as 293A-mIL12aIL2-IL12bGMCSF.

6.4 Expression and Purification of Proteins

The constructed cells 293 A-mIL12aIL2-IL12bGMCSF expressing mIL12aIL2-IL12bGMCSF were passaged into a 15 cm culture dish. After the cells were confluent, the culture medium was replaced to 30 ml of CDM4HEK293 to continue the culture for 5 days. The supernatant was then collected, filtered through a 0.45 μm filter, and concentrated by ultrafiltration with 50 kd of AMICON ULTRA-15. The concentrated protein solution obtained was purified with nickel-chelated magnetic beads (purchased from Beaver Biosciences Inc.) according to the instructions. The purified protein solution obtained was then ultrafiltered through an AMICON ULTRA-0.5 ultrafiltration tube, with the buffer being replaced to PBS. The finally obtained protein solution was tested with an IL12p70 ELISA kit for the protein concentration. After adjusting the protein concentration to 2 μg/μl with PBS, the protein solution was dispensed and stored at −20° C.

### Example 7. Effect of the Combination Treatment of mIL12aIL2-IL12bGMCSF and PD1 Antibody on the Growth of Melanoma in Mice

2×10⁵ digested and cultured mouse melanoma cells (B16) were injected subcutaneously into the right side of the body of C57BL/6 mice (6-10-week-old, female), and treatment was initiated when the long diameter of the tumor reached 6-8 mm.

25 μl of the protein solution prepared in Example 6 was taken and added into 50 μl of glycerol, and mixed well immediately with a pipette tip to avoid air bubbles to obtain an injection preparation. The formulated injection solution was drawn with a 29G insulin syringe and slowly injected into the tumor, leaving the needle in place for a little time after the injection to reduce the overflow of the solution. The injected mice were returned to their cages, and the tumor growth of the mice was recorded. This experiment was divided into 3 groups: 1. no injection group, 2. protein solution injection group, 3. protein solution injection and PD1 antibody treatment group (i.e., combined treatment group). Wherein, in the combined treatment group, 200 μg of PD1 antibody (specifically, BioXcell Inc., InVivoMAb anti-mouse PD-1 (CD279), Item No. BE0146) was injected intraperitoneally into the mice every 3 days starting from the second day after the injection of the protein solution, for a total of 4 injections. Finally, the tumor growth in each group of mice was compared. The results were shown in FIG. 4, from which it can be seen that the combined treatment of mIL12aIL2-IL12bGMCSF and PD1 antibody significantly inhibited the growth of tumor.

### Example 8. Effect of the Combination Treatment of mIL12bIL12aIL2DiaNHS76F8GMCSF and PD1 Antibody on the Growth of Breast Cancer in Mice

2×10⁵ digested and cultured mouse breast cancer cells (4T1) were injected subcutaneously into the right side of the body of Balb/c mice (6-10-week-old, female), and treatment was initiated when the long diameter of the tumor reached 6-8 mm.

25 μl of the protein solution prepared in Example 3 was taken and adjusted with PBS to a total volume of 200 μl. The protein solution was drawn with a 29G insulin syringe and injected into the tail vein of the mice once a day, for a total of 5 injections. The injected mice were returned to their cages, and the tumor growth of the mice was recorded. This experiment was divided into 3 groups: 1. no injection group, 2. protein solution injection group, 3. protein solution injection and PD1 antibody treatment group (i.e., combined treatment group). Wherein, in the combined treatment group, 200 μg of PD1 antibody (specifically, BioXcell Inc., InVivoMAb anti-mouse PD-1 (CD279), Item No. BE0146) was injected intraperitoneally into the mice every 3 days starting from the second day after the completion of 5 injections of the protein solution, for a total of 4 injections. Finally, the tumor growth in each group of mice was compared. The results were shown in FIG. 5, from which it can be seen that the combined treatment of mIL12bIL12aIL2DiaNHS76F8GMCSF and PD1 antibody significantly inhibited the growth of tumor.

### Example 9. Effect of the Combination Treatment of mIL12bIL12aIL2DiaNHS76F8GMCSF and PD1 Antibody on the Growth of Lung Cancer in Mice

2×10⁵ digested and cultured mouse lung cancer cells (LLC) were injected subcutaneously into the right side of the body of C57BL/6 mice (6-10-week-old, female), and treatment was initiated when the long diameter of the tumor reached 6-8 mm.

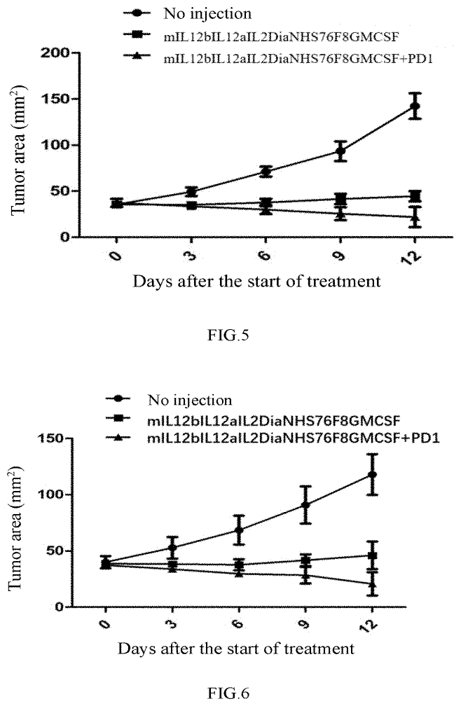

25 μl of the protein solution prepared in Example 3 was taken and adjusted with PBS to a total volume of 200 μl. The protein solution was drawn with a 29G insulin syringe and injected into the tail vein of the mice once a day, for a total of 3 injections. The injected mice were returned to their cages, and the tumor growth of the mice was recorded. This experiment was divided into 3 groups: 1. no injection group, 2. protein solution injection group, 3. protein solution injection and PD1 antibody treatment group (i.e., combined treatment group). Wherein, in the combined treatment group, 200 μg of PD1 antibody (specifically, BioXcell Inc., InVivoMAb anti-mouse PD-1 (CD279), Item No. BE0146) was injected intraperitoneally into the mice every 3 days starting from the second day after the completion of 5 injections of the protein solution, for a total of 4 injections. Finally, the tumor growth in each group of mice was compared. The results were shown in FIG. 6, from which it can be seen that the combined treatment of mIL12bIL12aIL2DiaNHS76F8GMCSF and PD1 antibody significantly inhibited the growth of tumor.

1. A pharmaceutical composition, comprising proteins and an immune checkpoint inhibitor, wherein the proteins comprise a fusion protein, and the fusion protein comprises cytokines IL12, IL2, and GMCSF.

2. The pharmaceutical composition according to claim 1, wherein the immune checkpoint inhibitor comprises inhibitors of PD1, PD-L1, and/or CTLA-4.

3. (canceled)

4. The pharmaceutical composition according to claim 1, wherein the proteins further comprise a targeting moiety.

5. The pharmaceutical composition according to claim 4, wherein the targeting moiety specifically recognizes and/or binds to a tumor-associated antigen.

6. The pharmaceutical composition according to claim 5, wherein the tumor-associated antigen is at least one selected from the following group consisting of an EDB domain of fibronectin, an EDA domain of fibronectin, and a necrotic region.

7. The pharmaceutical composition according to claim 4, wherein the targeting moiety comprises an antibody or an antigen binding fragment thereof.

8. The pharmaceutical composition according to claim 4, wherein the targeting moiety comprises an amino acid sequence as set forth in any one of the following groups: SEQ ID NOs. 1 to 15.

9. (canceled)

10. The pharmaceutical composition according to claim 1, wherein the proteins are single-stranded proteins comprising an amino acid sequence as set forth in any one of the following groups: SEQ ID NOs. 27 to 52.

11. The pharmaceutical composition according to claim 1, wherein the proteins are dimers composed of a first polypeptide chain and a second polypeptide chain, and the first polypeptide chain is different from the second polypeptide chain.

12. The pharmaceutical composition according to claim 11, wherein the first polypeptide chain comprises IL12a, and the second polypeptide chain comprises IL12b.

13. The pharmaceutical composition according to claim 11, wherein the IL2 or a functional fragment thereof is located in the first polypeptide chain or the second polypeptide chain, and
wherein the GMCSF or a functional fragment thereof is located in the first polypeptide chain or the second polypeptide chain.

14. The pharmaceutical composition according to claim 11, wherein the IL2 or a functional fragment thereof is located in the first polypeptide chain or the second polypeptide chain,
wherein the GMCSF or a functional fragment thereof is located in the first polypeptide chain or the second polypeptide chain, and
wherein one or more of the targeting moieties are each independently located in the first polypeptide chain or the second polypeptide chain.

15-16. (canceled)

17. The pharmaceutical composition according to claim 11, wherein,
a) the first polypeptide chain comprises an amino acid sequence as set forth in SEQ ID NO. 53, and the second polypeptide chain comprises an amino acid sequence as set forth in SEQ ID NO. 57; or
b) the first polypeptide chain comprises an amino acid sequence as set forth in SEQ ID NO. 54, and the second polypeptide chain comprises an amino acid sequence as set forth in SEQ ID NO. 57; or
c) the first polypeptide chain comprises an amino acid sequence as set forth in SEQ ID NO. 53, and the second polypeptide chain comprises an amino acid sequence as set forth in SEQ ID NO. 58; or
d) the first polypeptide chain comprises an amino acid sequence as set forth in SEQ ID NO. 54, and the second polypeptide chain comprises an amino acid sequence as set forth in SEQ ID NO. 58; or
e) the first polypeptide chain comprises an amino acid sequence as set forth in SEQ ID NO. 55, and the second polypeptide chain comprises an amino acid sequence as set forth in SEQ ID NO. 59; or
f) the first polypeptide chain comprises an amino acid sequence as set forth in SEQ ID NO. 56, and the second polypeptide chain comprises an amino acid sequence as set forth in SEQ ID NO. 60.

18-19. (canceled)

20. The pharmaceutical composition according to claim 11, wherein,
1) the first polypeptide chain comprises an amino acid sequence as set forth in SEQ ID NO. 66, and the second polypeptide chain comprises an amino acid sequence as set forth in SEQ ID NO. 61; or
2) the first polypeptide chain comprises an amino acid sequence as set forth in SEQ ID NO. 66, and the second polypeptide chain comprises an amino acid sequence as set forth in SEQ ID NO. 62; or
3) the first polypeptide chain comprises an amino acid sequence as set forth in SEQ ID NO. 66, and the second polypeptide chain comprises an amino acid sequence as set forth in SEQ ID NO. 63; or
4) the first polypeptide chain comprises an amino acid sequence as set forth in SEQ ID NO. 67, and the second polypeptide chain comprises an amino acid sequence as set forth in SEQ ID NO. 61; or
5) the first polypeptide chain comprises an amino acid sequence as set forth in SEQ ID NO. 68, and the second polypeptide chain comprises an amino acid sequence as set forth in SEQ ID NO. 62; or
6) the first polypeptide chain comprises an amino acid sequence as set forth in SEQ ID NO. 69, and the second polypeptide chain comprises an amino acid sequence as set forth in SEQ ID NO. 63; or
7) the first polypeptide chain comprises an amino acid sequence as set forth in SEQ ID NO. 70, and the second polypeptide chain comprises an amino acid sequence as set forth in SEQ ID NO. 64; or
8) the first polypeptide chain comprises an amino acid sequence as set forth in SEQ ID NO. 71, and the second polypeptide chain comprises an amino acid sequence as set forth in SEQ ID NO. 64; or
9) the first polypeptide chain comprises an amino acid sequence as set forth in SEQ ID NO. 72, and the second polypeptide chain comprises an amino acid sequence as set forth in SEQ ID NO. 65.

21. A reagent kit, comprising the pharmaceutical composition according to claim 1.

22-27. (canceled)

28. A method for treating a tumor, comprising administering to a subject in need thereof the pharmaceutical composition according to claim 1 or a reagent kit comprising the pharmaceutical composition.

29. The method according to claim 28, wherein the administering comprises administering the proteins first, and then administering the immune checkpoint inhibitor.

30. The method according to claim 28, wherein the administering comprises intra-tumoral injection, intravenous injection, or subcutaneous injection.

31. The method according to claim 28, wherein the tumor comprises a solid tumor.

32. The method according to claim 28, wherein the tumor comprises melanoma, breast cancer, and/or lung cancer.

33-34. (canceled)
